SEQUENCE LISTING (4 sequences)

SEQ ID NO: 1 - DNA, 25 bp, Bacteriophage T7
aattctaatacgactcactataggg

SEQ ID NO: 2 - DNA, 31 bp, Artificial
  Random-T7 Primer
aattctaatacgactcactatagggnnnnnn

SEQ ID NO: 3 - DNA, 24 bp, Artificial
  Template-Switching Oligonucleotide
tgctgcgagaagacgacagaaggg

SEQ ID NO: 4 - DNA, 21 bp, Artificial
  5′-Anchor Primer
tgctgcgagaagacgacagaa

(19) United States
(12) Patent Application Publication
(10) Pub. No.: US 2004/0006033 A1
(43) Pub. Date: Jan. 8, 2004

(54) Methods for identifying low-abundance polynucleotides and related compositions

(76) Inventor: York Yuan-Yuan Zhu, Palo Alto, CA (US)

Correspondence Address: Emily M Haliday, Quine Intellectual Property Law Group, PO Box 458, Alameda, CA, 94501, US

(21) Appl. No.: 10/343,345

(22) PCT Filed: Aug. 6, 2001
(86) PCT No.: PCT/US01/24730

(51) Int. Cl. A61K 48/00

(52) U.S. Cl. 514/44

(57) ABSTRACT

This invention provides novel methods for producing a plurality of polynucleotides prepared from a polynucleotide sample and the plurality of polynucleotides so produced. In one embodiment, the plurality of polynucleotides is prepared by subtractive hybridization between test and reference polynucleotide samples and is substantially enriched in sequences that are either not present in the reference polynucleotide sample or are present in the reference polynucleotide sample in substantially lower concentration than in the test polynucleotide sample. The plurality of polynucleotides is also substantially enriched in low-abundance sequences, relative to the test polynucleotide sample. The invention also provides kits useful in the methods of the invention and for using the polynucleotides produced thereby. The polynucleotides are useful in a wide variety of applications, such as cloning, expression, and hybridization studies.

### RELATED APPLICATION INFORMATION

This application is a continuation-in-part of application Ser. No. 09/632,898 (filed Aug. 7, 2000) and a continuation-in-part of Application No. 06/288,777 (filed May 4, 2001), both of which are incorporated by reference herein in their entirety.

### FIELD OF THE INVENTION

This invention relates to methods and kits for selecting polynucleotide pools from a sample and the selected polynucleotide pools produced thereby. In particular, this invention provides a method for preparing a polynucleotide pool enriched in high-abundance sequences relative to the sample and a subtractive hybridization reaction for using such a polynucleotide pool to prepare a polynucleotide pool enriched in low-abundance sequences. The invention also provides a general subtractive hybridization method that produces a subtracted polynucleotide pool from a test and reference polynucleotide sample.

### BACKGROUND OF THE INVENTION

Biologically active proteins have been the subject of intense research as candidates for therapeutic, diagnostic, and other applications. The first step in these efforts is typically the cloning of the gene encoding the protein from messenger RNA (mRNA). The mRNA of human and other mammalian cells can be divided into three frequency classes: (1) high-abundance sequences, which represent about 10-20% of the total mRNA population; (2) medium-abundance sequences, which represent about 40-45% of mRNA, and (3) low-abundance sequences, which represent another 40-45% of mRNA. Many genes encoding proteins with important regulatory functions, such as hormones and their receptors, are expressed at a low level and the corresponding transcripts fall into the low-abundance class of sequences.

Efforts to clone low-abundance sequences have employed normalized cDNA libraries in which the frequencies of all clones in the library are within a narrow range. However, this approach does not address the loss of low-abundance sequences in the process of generating the cDNA library, which preferentially clones high- and medium-abundance sequences as well as shorter sequences. A method that facilitated the selection of a pool low-abundance polynucleotides from an mRNA population and that provided a means to produce large quantities of such sequences, without the losses that accompany cloning, would greatly assist research aimed at identifying important regulatory proteins.

Of particular interest would be a method that facilitated the identification of low-abundance polynucleotides that are differentially expressed between two samples. Efforts to identify such polynucleotides have generally used some variation of subtractive hybridization. In this technique, the two samples are typically two mRNA samples, with the sample containing the transcripts of interest denoted the “test” sample and the sample that lacks the transcripts interest termed the “reference” sample or the “driver.” For example, the test sample might consist of mRNA from a tumor cell and the reference sample, mRNA from a normal cell. Both samples are typically first converted into cDNA samples, which are then hybridized. Polynucleotides common to both samples form hybrids, which are removed in some fashion, leaving single-stranded polynucleotides that differ between the two samples. Although traditional subtractive hybridization techniques have been successful in some case, the two major drawbacks of these techniques are that they are not suitable for identifying rare mRNAs and they generally do not yield full-length transcripts. Full-length transcripts are essential for expressing the encoded protein and carrying out functional studies.

In traditional subtractive hybridization, the mRNA samples are converted to cDNAs and then digested with a frequently cutting restriction enzyme to ensure that each cDNA molecule is “sticky ended.” Adaptors are ligated onto the sticky ends, and the fragments are then cloned into a vector. Digestion with a frequently cutting restriction enzyme is necessary to ensure that the resulting cDNA libraries are as representative as possible of the original mRNA samples, but has the undesirable effect of producing truncated cDNA clones. Such clones can be used to probe additional libraries in an effort to obtain a full-length clone, which is, at best, laborious and often futile in the case of low-abundance transcripts, which are typically under-represented or absent in all available libraries.

Ideally, methods aimed at identifying low-abundance polynucleotides, particularly those that differ between samples, would be capable of replicating a broad range of transcripts without prior cloning into vectors and without requiring knowledge of sequence. Preferably, the low-abundance polynucleotides produced by such methods would be representative of the full-length transcripts (e.g., full-length cDNA clones). Finally, a method that facilitated the identification of low-abundance polynucleotides that differed between two samples would be of particular interest.

### SUMMARY OF THE INVENTION

The present invention provides a subtractive hybridization method for identifying one or more polynucleotides in a test sample that are absent from, or less abundant in, a reference sample. The method is particularly useful for identifying low-abundance sequences that are differentially expressed between different samples. In preferred embodiments, the test and reference polynucleotide samples are selected from one of the following:

(a) mRNA from a first cell or tissue and mRNA from a second, different cell or tissue;

(b) mRNA from a cell or tissue at a first stage of differentiation or development and mRNA from the cell or tissue at a second, different stage of differentiation or development;

(c) mRNA from a cell or tissue treated with an active agent and mRNA from a cell or tissue that is untreated or treated with a second, different active agent; and

(d) mRNA from a normal cell or tissue and mRNA from a diseased cell or tissue, e.g., wherein the disease is an infectious disease or a cancer.

The method uses high-abundance enriched polynucleotide strands of, or prepared from, a pool of test or reference polynucleotides that is enriched in high-abundance polynucleotide sequences relative to a test or reference polynucleotide sample as driver in a first subtractive hybridization reaction to “remove” high-abundance sequences from the hybridization mixture. In one embodiment, the high-abundance enriched polynucleotide strands are of, or prepared from a pool of test polynucleotides. Preferably, the high-abundance enriched polynucleotide strands are high-abundance enriched antisense polynucleotide strands. For example, if the test or reference polynucleotide sample is an mRNA sample, the high-abundance enriched antisense polynucleotide strands are preferably antisense RNA molecules.

This first subtractive hybridization entails contacting the high-abundance enriched polynucleotide strands with test polynucleotide strands of, or prepared from, the test polynucleotide sample under hybridization conditions to form a first hybridization mixture. The test polynucleotide strands are preferably sense test polynucleotide strands, such as mRNA molecules. This reaction produces unhybridized test polynucleotide strands that are enriched in low-abundance polynucleotide sequences relative to the test polynucleotide sample.

Polynucleotide strands for use in a second subtractive hybridization reaction are then synthesized from the unhybridized test polynucleotide strands, thereby producing low-abundance enriched test polynucleotide strands. These low-abundance enriched test polynucleotide strands are preferably antisense test polynucleotide strands, such as antisense cDNA strands. The driver for this second subtractive hybridization reaction is low-abundance-enriched reference polynucleotide stands of, or prepared from, a reference pool of polynucleotides that is enriched in low-abundance polynucleotide sequences relative to the reference polynucleotide sample. The low-abundance enriched reference polynucleotide strands are preferably sense reference polynucleotide strands, e.g., sense cDNA strands.

This second hybridization reaction “removes” sequences present in both samples (in roughly similar amounts), leaving differentially expressed sequences unhybridized. The reaction is carried out by contacting the low-abundance enriched reference polynucleotide strands with the low-abundance enriched test polynucleotide strands under hybridization conditions to form a second hybridization mixture, thereby producing hybrid duplexes, unhybridized low-abundance enriched reference polynucleotide strands, and unhybridized low-abundance enriched test polynucleotide strands.

The hybrid duplexes, which represent common sequences are removed or digested, and “test-specific duplexes” are produced from the unhybridized low-abundance enriched test polynucleotide strands. Preferably, the hybrid duplexes are digested with at least one enzyme, such as, e.g., a restriction endonuclease, and the test-specific duplexes are produced from the unhybridized antisense test polynucleotide strands by amplification.

In preferred embodiments, the antisense test polynucleotide strands are synthesized from the unhybridized sense test polynucleotide strands using a first antisense primer or a first antisense primer complex. In a variation of this embodiment, the first antisense primer or first antisense primer complex includes: (a) a sequence that binds to a primer site in the unhybridized sense test polynucleotide strands; (b) a first restriction site 5′ of the sequence of (a), wherein the first restriction site is cleaved by a restiction endonuclease that cleaves double-stranded polynucleotides, but leaves single-stranded polynucleotides substantially intact; and (c) a first universal primer site 5′ of the restriction site of (b). In this variation of the invention, the antisense test polynucleotide strands are preferably synthesized from the unhybridized sense test polynucleotide strands using a first antisense primer, and the first universal primer site of (c) includes a second restriction site, wherein said second restriction site is different from the first restriction site. In addition, the sense reference polynucleotide strands preferably include a third restriction site previously added to the 5′ ends of the sense reference polynucleotide strands, wherein the third restriction site is cleaved by a restriction endonuclease that cleaves double-stranded polynucleotides, but leaves single-stranded polynucleotides substantially intact.

After introducing suitable restriction sites into the polynucleotide strands that form hybrid duplexes, digestion of the hybrid duplexes can conveniently be accomplished by (a) treating the second hybridization mixture with an enzyme that renders single-stranded portions of the hybrid duplexes double-stranded, thereby producing double-stranded first, second, and third restriction sites in the hybrid duplexes; (b) adding a second universal primer site to the 3′ ends of the polynucleotide strands in the second hybridization mixture; and (c) treating the second hybridization mixture with one or more restriction endonuclease(s), wherein the restriction endonuclease(s) cleave(s) the hybrid duplexes at the double-stranded first and third restriction sites, but leaves the antisense test polynucleotide strands and the sense reference polynucleotide strands substantially intact.

The methods of the invention generally includes cloning one or more of the test specific duplexes into a vector. In particular, such methods allow the construction of a “test specific” cDNA library. This library is superior to libraries produced by other subtractive hybridization techniques in that it will generally contain low-abundance sequences that are lost using conventional techniques. In a preferred variation of this embodiment, the cloned test-specific duplex encodes a polypeptide, and the vector is an expression vector. The methods of the invention can further include introducing the expression vector into a host cell and expressing the protein encoded by the test specific duplexes.

In one embodiment, the test-specific duplexes are synthesized from the unhybridized antisense test polynucleotide strands using a first antisense primer complex, the method additionally includes synthesizing one or more antisense RNA molecules from the test-specific duplexes. Antisense RNA molecules produced in this manner can be introduced into a cell for research or therapeutic purposes.

The test-specific duplex produced according to the methods of the invention, or a polynucleotide produced directly or indirectly therefrom, can also be used in a hybridization reaction. Such polynucleotides can be labeled with a detectable label and/or attached to a substrate to produce a polynucleotide array. If desired, one or more of the test-specific duplexes can be amplified. In a preferred embodiment, this amplification is carried out using one or more gene-specific primers. Accordingly, the methods of the invention encompass each of these applications of the test-specific duplexes.

In another embodiment, the invention provides a method for functionally isolating single-stranded polynucleotides in a mixture of single- and double-stranded polynucleotides. This method entails contacting a mixture of single- and double-stranded polynucleotides with one or more restriction endonucleases under conditions sufficient to allow digestion of double-stranded polynucleotides to a form that cannot serve as a template for a nucleotide synthesis reaction that uses the single-stranded polynucleotides as a template. Preferably, the restriction endonuclease(s) cleave(s) a primer site from double-stranded polynucleotides in said mixture. After restriction digestion, uncleaved single-stranded polynucleotides in the mixture are preferably converted to double-stranded polynucleotides by amplification.

Another aspect of the invention is a plurality of polynucleotides prepared by subtractive hybridization between test and reference polynucleotide samples, wherein the plurality of polynucleotides includes at least 10³ different polynucleotides. The plurality of polynucleotides is substantially enriched in sequences that are: (a) either not present in the reference polynucleotide sample or are present in the reference polynucleotide sample in substantially lower concentration than in the test polynucleotide sample, and (b) low-abundance sequences, relative to the test polynucleotide sample. Each of the polynucleotides in the plurality of polynucleotides can include an RNA promoter sequence and a universal primer site. In preferred embodiments, these polynucleotides are double-stranded cDNA or antisense RNA.

The invention also provides kits useful for performing the methods of the invention and/or using the plurality of polynucleotides of the invention. A first kit includes an antisense primer or antisense primer complex and instructions for performing the general subtractive hybridization method of the invention. The antisense primer or antisense primer complex includes: (a) a sequence that binds to a primer site; (b) a first restriction site 5′ of the sequence of (a), wherein the first restriction site is cleaved by a restiction endonuclease that cleaves double-stranded polynucleotides, but leaves single-stranded polynucleotides substantially intact; and (c) a first universal primer site 5′ of the restriction site of (b).

A second kit includes: a plurality of polynucleotides of the invention; an antisense primer complex comprising a antisense primer operably linked to an RNA promoter sequence, wherein the RNA promoter sequence is 5′ of the antisense primer; and a sense primer. A third kit includes: a plurality of polynucleotides of the invention, and an RNA polymerase capable of transcribing antisense RNA from the plurality of polynucleotides.

The invention also provides a method for preparing a selected polynucleotide pool from a polynucleotide sample. In a preferred embodiment, the selected polynucleotide pool is enriched in one or more high-abundance polynucleotides relative to the polynucleotide sample. The method entails synthesizing first antisense polynucleotide strands from sense polynucleotides of, or prepared from, the polynucleotide sample using an antisense primer complex. The antisense primer complex includes an antisense primer operably linked to an RNA promoter sequence, such that the RNA promoter sequence is 5′ of the antisense primer. Next, a universal primer site is added to the 3′ ends of the first antisense polynucleotide strands. The resultant first antisense polynucleotide strands are then diluted to substantially eliminate at least some low-abundance first antisense polynucleotide strands. After dilution, first double-stranded polynucleotides are produced from the remaining first antisense polynucleotide strands. The first double-stranded polynucleotides are enriched in high-abundance polynucleotide sequences relative to the polynucleotide sample.

In a preferred embodiment of the method, the polynucleotide sample is an mRNA sample, the first antisense polynucleotide strands are first antisense cDNA strands, and the first double-stranded polynucleotides are first double-stranded cDNA molecules. The synthesis of first antisense cDNA strands can be primed using a random primer or an oligonucleotide-dT primer. The universal primer site can be added to the 3′ end of the first antisense cDNA strands by template switching, oligonucleotide-tailing, or ligation. The RNA promoter sequence is conveniently one that is recognized by a bacteriophage RNA polymerase, such as T7, T3, or SP6 polymerase.

Preferably, the first double-stranded polynucleotides are produced by amplifying the first antisense polynucleotide strands remaining after dilution, and the amplification is carried out using a universal primer that hybridizes to the universal primer site as the 5′ primer and using the antisense primer complex as the 3′ primer. Most preferably, the amplification is performed by enhanced polymerase chain reaction. This reaction produces a pool of double-stranded polynucleotides that are enriched in high-abundance sequences relative to the original polynucleotide sample. The method optionally includes synthesizing first antisense RNA molecules from the first double-stranded polynucleotides. This pool of antisense RNA molecules is enriched in high-abundance sequences and can therefore be used as a “driver” in subtractive hybridization.

The invention also provides a method of using antisense polynucleotide strands, preferably the high-abundance-enriched antisense RNA molecules prepared as described above, to produce a selected polynucleotide pool from a polynucleotide sample. In a preferred embodiment, the selected polynucleotide pool is enriched in one or more low-abundance polynucleotides relative to the polynucleotide sample. The method entails hybridizing first antisense polynucleotide strands to sense polynucleotide strands of, or prepared from, a polynucleotide sample under hybridization conditions. Preferably, the molar ratio of the first antisense polynucleotide strands to the other polynucleotides in the hybridization mixture is between about 1 and about 100 to 1.

The resulting hybridization mixture includes unhybridized sense polynucleotide strands that are enriched in low-abundance polynucleotide sequences relative to the polynucleotide sample. Second antisense polynucleotide strands are synthesized from the unhybridized sense polynucleotide strands using an antisense primer or an antisense primer complex. The antisense primer complex includes an antisense primer operably linked to an RNA promoter sequence, such that the RNA promoter sequence is 5′ of the antisense primer. An antisense primer complex is preferably employed if it is desirable to produce a pool of selected polynucleotides that each include an RNA promoter to facilitate the synthesis of antisense RNA from the selected polynucleotides.

Next, a universal primer site is added to the 3′ ends of the second antisense polynucleotide strands. Second double-stranded polynucleotides are then produced from the second antisense polynucleotide strands. This pool of polynucleotides is enriched in low-abundance polynucleotide sequences relative to the polynucleotide sample.

In a preferred embodiment of the method, the polynucleotide sample is an mRNA sample, the sense polynucleotide strands are mRNA molecules, the second antisense polynucleotide strands are second antisense cDNA strands, and the second double-stranded polynucleotides are second double-stranded cDNA molecules. The synthesis of second antisense cDNA strands can be primed using an oligonucleotide-dT primer. The universal primer site can be added to the 3′ end of the second antisense cDNA strands by template switching, oligonucleotide-tailing, or ligation. If an antisense primer complex is employed, the RNA promoter sequence is conveniently one that is recognized by a bacteriophage RNA polymerase, such as T7, T3, or SP6 polymerase.

Preferably, the second double-stranded polynucleotides are produced by amplifying the second antisense polynucleotide strands, and the amplification is carried out using a universal primer that hybridizes to the universal primer site as the 5′ primer and using the antisense primer or antisense primer complex as the 3′ primer. Most preferably, the amplification is performed by enhanced polymerase chain reaction. This reaction produces a pool of double-stranded polynucleotides that is enriched in low-abundance sequences relative to the original polynucleotide sample. If an antisense primer complex is used to produce second double-stranded polynucleotides, these polynucleotides contain an RNA promoter. In this case, the method can optionally include synthesizing antisense RNA molecules from the second double-stranded polynucleotides.

In preferred embodiments of the methods of the invention, the universal primer and/or the antisense primer or antisense primer complex each comprise a restriction site. The methods of the invention can optionally include cloning one or more of the second double-stranded (low-abundance-enriched) polynucleotides into a vector. In particular, such methods allow the construction of a “normalized” cDNA library. This library is superior to normalized libraries produced by other techniques in that the copy numbers of the cDNAs in the library vary by much less than in the original polynucleotide sample; e.g., highly representative cDNA libraries can be produced in which cDNA copy numbers vary by no more than an order of magnitude. In a preferred variation of this embodiment, the cloned double-stranded polynucleotide encodes a polypeptide, and the vector is an expression vector. The methods of the invention can further include introducing the expression vector into a host cell and expressing the protein encoded by the cloned double-stranded polynucleotide.

The double-stranded polynucleotides produced according to the methods of the invention, or a polynucleotide produced directly or indirectly therefrom, can also be used in a hybridization reaction. Such polynucleotides can be labeled with a detectable label and/or attached to a substrate to produce a polynucleotide array. If desired, one or more of the second double-stranded polynucleotides can be amplified. In a preferred embodiment, this amplification is carried out using one or more gene-specific primers. Accordingly, the methods of the invention encompass each of these applications of these polynucleotides.

In an alternative embodiment, the method for preparing a selected polynucleotide pool from a polynucleotide sample is carried out by synthesizing first antisense polynucleotide strands from sense polynucleotides of, or prepared from, the polynucleotide sample and diluting the first antisense polynucleotide strands to substantially eliminate at least some low-abundance first antisense polynucleotide strands. First double-stranded polynucleotides are then produced from the remaining first antisense polynucleotide strands. These first double-stranded polynucleotides are enriched in high-abundance polynucleotide sequences relative to the polynucleotide sample. They are used to produce second antisense polynucleotide strands, which are then contacted with sense polynucleotide strands of, or prepared from, the polynucleotide sample under hybridization conditions. The resulting hybridization mixture includes unhybridized sense polynucleotide strands that are enriched in low-abundance polynucleotide sequences relative to the polynucleotide sample. Third antisense polynucleotide strands are synthesized from the unhybridized sense polynucleotide strands, and second double-stranded polynucleotides are produced from the third antisense polynucleotide strands. These second double-stranded polynucleotides make up a selected polynucleotide pool that is enriched in low-abundance polynucleotide sequences.

Another aspect of the invention is a plurality of polynucleotides prepared from a polynucleotide sample, wherein the plurality of polynucleotides includes at least 10³ different polynucleotides and is either substantially enriched in high-abundance polynucleotide sequences relative to the polynucleotide sample or substantially enriched in low-abundance polynucleotide sequences relative to the polynucleotide sample. Each of the polynucleotides in the plurality of polynucleotides preferably includes a RNA promoter sequence and a universal primer site. In preferred embodiments, these polynucleotides are double-stranded cDNA or antisense RNA.

The invention also provides kits useful for performing the methods of the invention and/or using the plurality of polynucleotides of the invention. A first kit includes: an antisense primer complex including an antisense primer operably linked to an RNA promoter sequence, wherein the RNA promoter sequence is 5′ of the antisense primer; a sense primer; and instructions for performing at least one of the above-described methods of the invention. A second kit includes: a plurality of polynucleotides of the invention; an antisense primer complex comprising a antisense primer operably linked to an RNA promoter sequence, wherein the RNA promoter sequence is 5′ of the antisense primer; and a sense primer. A third kit includes: a plurality of polynucleotides of the invention, and an RNA polymerase capable of transcribing antisense RNA from the plurality of polynucleotides.

### BRIEF DESCRIPTION OF THE DRAWINGS

FIGS. 1A-1B shows a schematic representation of a preferred embodiment of a selection method of the invention that is useful for preparing a pool of polynucleotides that is enriched in high-abundance sequences relative to a polynucleotide sample. This embodiment, which is described in detail in Example 4, can be used to prepare high-abundance enriched antisense RNA driver for use in the first subtractive hybridization reaction of the general subtractive hybridization method of the invention.

FIGS. 2A-2C shows a schematic representation of a preferred embodiment of a second selection method of the invention, which is useful for preparing a pool of polynucleotides that is enriched in low-abundance sequences relative to a polynucleotide sample. This embodiment, which is described in detail in Example 4, can be used to prepare low-abundance enriched sense cDNA driver for use in the second subtractive hybridization reaction of the general subtractive hybridization method of the invention.

FIGS. 3A-3D shows a schematic representation of a preferred embodiment of the general subtractive hybridization method of the invention. The steps of this embodiment are described in detail in Example 4C.

### DETAILED DESCRIPTION

The invention includes a novel subtractive hybridization method that allows the identification of low-abundance polynucleotides that are present in one polynucleotide sample but absent (or substantially reduced) in a second polynucleotide sample. This method relies on two subtractive hybridization reactions. In the first, excess high-abundance polynucleotide strands from a test or reference polynucleotide sample are contacted with polynucleotide strands from the test sample, thereby removing high-abundance polynucleotide sequences. The resultant mixture is used to produce low-abundance enriched polynucleotide strands corresponding to the test sample. In the second hybridization reaction, the low-abundance enriched polynucleotide strands derived from the test sample are contacted with excess low-abundance polynucleotide strands from the reference sample to block those low-abundance polynucleotide sequences that are present in both samples. The remaining single-stranded low-abundance polynucleotides unique to the test sample are then rendered double-stranded and can be used for subsequent analyses or manipulations, typically after cloning into vectors. This method is particularly useful in studies aimed, for example, at identifying low-abundance disease-related genes, which are difficult to identify using conventional subtractive hybridization technologies.

This strategy depends on the availability of pools of polynucleotides that are enriched in high- and low-abundance polynucleotide sequences, relative to a polynucleotide sample from which they were derived (i.e., relative to the “starting polynucleotide sample”). A preferred embodiment employs novel methods for generating such polynucleotide pools. In particular, high-abundance polynucleotides are selected by exploiting the loss of low-abundance polynucleotides that occurs upon dilution. Low-abundance polynucleotides can then be selected by subtractive hybridization between the high-abundance polynucleotide-enriched pool and sample polynucleotides.

The methods described herein can be used to replicate a broad range of polynucleotides without prior cloning into vectors and without sequence information. If desired, polynucleotides that represent full-length mRNA transcripts can be produced. Polynucleotides produced according to these methods are useful in a wide variety of applications, such as cloning, expression, and hybridization studies.

I. Definitions

The term “polynucleotide” refers to a deoxyribonucleotide or ribonucleotide polymer, and unless otherwise limited, includes known analogs of natural nucleotides that can function in a similar manner to naturally occurring nucleotides.

The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA.

The term “polynucleotide” encompasses double-stranded polynucleotides, as well as single-stranded molecules. Double-stranded polynucleotides that encode a protein contain a “sense” polynucleotide strand hydrogen-bonded to an “antisense” polynucleotide strand. The sense polynucleotide strand is the strand whose nucleotide sequence, when translated, provides the amino acid sequence of the encoded protein. The term “sense polynucleotide strand” refers, for example, to the sense strands of double-stranded DNA molecules, as well as to mRNA. The antisense polynucleotide strand is complementary to the sense polynucleotide strand. Examples of antisense polynucleotide strands include the antisense strands of double-stranded DNA molecules (e.g., antisense cDNA strands) and antisense RNA molecules. In double-stranded polynucleotides, the polynucleotide strands need not be coextensive (i.e, a double-stranded polynucleotide need not be double-stranded along the entire length of both strands).

The term “duplex” is used herein to refer to a double-stranded polynucleotide. A “hybrid duplex” is a duplex formed between two polynucleotide strands in a subtractive hybridization reaction, wherein each strand is derived from a different polynucleotide sample.

As used herein, the term “complementary” refers to the capacity for precise pairing between two nucleotides. I.e., if a nucleotide at a given position of a polynucleotide is capable of hydrogen bonding with a nucleotide of another polynucleotide, then the two polynucleotides are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH 7.

“Specific hybridization” refers to the binding of a polynucleotide to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

Hybridization converts a single-stranded polynucleotide into a double-stranded polynucleotide, which can prevent the formerly single-stranded polynucleotide from serving as a target for hybridization or as a template for synthesis of additional polynucleotide strands. Accordingly, hybridization is said to “block” the formerly single-stranded polynucleotide.

As used with reference to polynucleotide strands, the term “unhybridized” refers to a polynucleotide that remains single-stranded after a hybridization reaction has been carried out under conditions where at least some polynucleotide strands hybridize to form double-stranded polynucleotides.

The term “driver” is used herein to refer to a particular type of polynucleotide strands added to a subtractive hybridization reaction to block complementary sequences present in the reaction mixture. Generally driver is added to the reaction mixture in molar excess to drive the hybridization.

The term “oligonucleotide” is used to refer to a polynucleotide that is relatively short, generally shorter than 200 nucleotides, more particularly, shorter than 100 nucleotides, most particularly, shorter than 50 nucleotides. Typically, oligonucleotides are single-stranded DNA molecules.

The term “selected polynucleotide pool” is used to describe a collection of polynucleotides that represents a subset of the polynucleotides present in a polynucleotide sample used to produce the selected polynucleotide pool. As used herein, this term describes the high- and low-abundance pools produced by dilution and by subtractive hybridization after dilution, respectively.

The term “subtracted polynucleotide pool” is used herein to refer to the polynucleotide pool produced by subtractive hybridization between polynucleotides derived from two different samples (e.g., using the general subtractive hybridization method of the invention). Thus, the “substracted polynucleotide pool” contains polynucleotides present in one sample and absent (or substantially reduced) in the other sample. When reference polynucleotides are subtracted from test polynucleotides, the polynucleotides of the subtracted polynucleotide pool are termed “test-specific” to indicate their presence in the test sample and substantial absence in the reference. Subtracted polynucleotide pools produced according to the general subtractive hybridization method of the invention are typically produced as a library of polynucleotides cloned into a vector (i.e., libraries of polynucleotide clones).

The term “primer” refers to an oligonucleotide that is capable of hybridizing (also termed “annealing”) with a polynucleotide and serving as an initiation site for nucleotide (RNA or DNA) polymerization.

An “antisense primer” is a primer that hybridizes with a nucleotide sequence present in a sense polynucleotide and that can serve as an initiation site for synthesis of an antisense polynucleotide.

A “sense primer” is a primer that hybridizes with a nucleotide sequence present in an antisense polynucleotide and that can serve as an initiation site for synthesis of a sense polynucleotide. As used herein, a sense primer has a sequence that enables it to be used with an antisense primer or antisense primer complex to amplify one or more target polynucleotide sequences.

A “universal primer” is one that hybridizes with a nucleotide sequence present in substantially all polynucleotides intended to serve as the template molecules for nucleotide polymerization.

A “gene-specific primer” is one that hybridizes with a nucleotide sequence present in or flanking a unique expressed sequence, allowing amplification of the unique expressed sequence, or a portion thereof, without substantial amplification of other sequences.

The term “primer site” refers to a region of a polynucleotide that is capable of hybridizing with a primer and serving as an initiation site for nucleotide (RNA or DNA) polymerization.

A “universal primer site” is primer site present in substantially all polynucleotides intended to serve as the template molecules for nucleotide polymerization.

The term “antisense primer complex” is used herein to denote an antisense primer operably linked to an oligonucleotide including an “RNA promoter sequence.” The latter sequence is one that provides a promoter in the correct orientation to serve as an initiation site for RNA polymerization.

As used herein, the term “operably linked” refers to a functional linkage between a control sequence (typically a promoter) and the linked sequence.

The term “abundance” is used to describe the number of copies of a polynucleotide in a polynucleotide sample. A polynucleotide present in a sample at greater than the median number of copies for a polynucleotide of the sample is said to be a “high-abundance polynucleotide.” A polynucleotide present in a sample at less than the median number of copies is said to be a “low-abundance polynucleotide.” The absolute number of copies of high- or low-abundance sequences varies, depending on the polynucleotide sample. In an mRNA sample, high-abundance sequences include mRNAs transcribed from so-called “housekeeping genes,” whereas low-abundance sequences include those encoding regulatory proteins, such as hormones, receptors, or other signaling and control molecules. Low-abundance mRNAs that can be selected for according to the methods of the invention typically account for less than about 1%, less than about 0.1%, less than 0.01%, or less than about 0.001% of the mRNA present in a cell. The methods of the invention can also be used to select the rarest of mRNAs, which account for on the order of only about 0.0000001% of the mRNA present in a cell. mRNA frequencies are typically estimated by screening a cDNA library with a probe that specifically hybridizes to an mRNA of interest, The number of positive clones divided by the total number of clones in the in the library, multiplied by 100%, gives the representation of the sequence in the library, which provides an estimate of mRNA frequency in the cells from which the library was produced.

A polynucleotide that is present at high abundance in a polynucleotide sample is said to be “highly represented” in the sample.

A pool of polynucleotides is said to be “enriched” in polynucleotides of a given type relative to a polynucleotide sample when such polynucleotides are present in a higher concentration in the pool than in the sample. This term is used herein to describe the products of a subtractive hybridization reaction as follows. If, for example, the subtractive hybridization reaction entails hybridizing a pool of high-abundance enriched polynucleotide strands selected from a sample with (non-enriched) polynucleotide strands of the same sample, the high-abundance sequences hybridize. Low-abundance sequences that are present in the sample remain unhybridized. Thus, the subtractive hybridization reaction produces unhybridized polynucleotide strands that are “enriched” in low-abundance polynucleotide sequences relative to the polynucleotide sample. Those of skill in the art understand that the concentration of low-abundance polynucleotide sequences present in the reaction mixture is the same after the reaction as before. However, the concentration of low-abundance sequences is higher in the pool of unhybridized polynucleotide strands than in the polynucleotide strands of the sample.

The phrase “high-abundance enriched polynucleotide strands” refers to a collection of polynucleotide strands that is enriched in high-abundance polynucleotide sequences relative to a polynucleotide sample.

The phrase “low-abundance enriched polynucleotide strands” refers to a collection of polynucleotide strands that is enriched in low-abundance polynucleotide sequences relative to a polynucleotide sample.

As used herein, “substantially enriched” means an enrichment of about 100-fold; i.e., a selected polynucleotide pool is substantially enriched in high- or low-abundance sequences if the concentrations of a plurality of high- or low-abundance sequences is at least about 100-fold higher in the selected polynucleotide pool relative to the original polynucleotide sample from which the pool was derived. For this purpose, enrichment can be estimated by hybridizing a labeled probe to the polynucleotide sample and to the selected polynucleotide pool and comparing the hybridization signal observed for each. For example, a Northern blot can be prepared from the polynucleotide sample and the selected polynucleotide pool and hybridized with a radioactively labeled probe, followed by autoradiography. The autoradiograph can be scanned using laser densitometry to quantitate the hybridization signal. Other techniques for determining the intensity of a hybridization signal, e.g, array-based methods, are well known and can be employed to assess enrichment of polynucleotide sequences in the present invention. “Fold enrichment” is calculated by dividing the hybridization signal observed for the selected polynucleotide pool by the hybridization signal observed for the polynucleotide sample from which the pool was selected.

Polynucleotides of a particular type (e.g., low-abundance polynucleotides) are said to be “substantially eliminated” if the concentration of such polynucleotides in a pool of polynucleotides is sufficiently reduced that the pool of polynucleotides can be used for applications wherein the presence of such polynucleotides is undesirable.

A polynucleotide is said to be “present in a reference polynucleotide sample in substantially lower concentration than in a test polynucleotide sample” if the abundance of the polynucleotide in the reference sample is at least about 100-fold less than in the in the test sample. The difference in abundance can be on the order of about 10³, about 10⁴, about 10⁵, and about 10⁶, or greater.

The phrase “polynucleotides of a polynucleotide sample” refers to the sample polynucleotides. The phrase “polynucleotides prepared from a polynucleotide sample” refers to polynucleotides produced from sample polynucleotides by RNA or DNA polymerization (e.g., reverse transcription, amplification, synthesis of antisense RNA, etc.) Polynucleotides are “produced directly” from sample polynucleotides when the sample polynucleotides serve as templates for RNA or DNA polymerization. Polynucleotides are “produced indirectly” from sample polynucleotides when more than one polymerization step is employed. Polynucleotides of, or prepared from, a sample and used as a starting material in any of the selection or subtraction methods of the invention are referred to herein as “starting polynucleotides.”

As used herein, the term “enhanced polymerase chain reaction” or “enhanced PCR” refers to a polymerase chain reaction capable of amplifying polynucleotide sequences of at least 10 kilobases (kb) in length.

The term “vector” is used herein to describe a DNA construct containing a polynucleotide. Such a vector can be propagated stably or transiently in a host cell. The vector can, for example, be a plasmid, a viral vector, or simply a potential genomic insert. Once introduced into a suitable host, the vector may replicate and function independently of the host genome, or may, in some instances, integrate into the host genome.

“Expression vector” refers to a DNA construct containing a polynucleotide molecule that is operably linked to a control sequence capable of effecting the expression of the polynucleotide in a suitable host. Exemplary control sequences include a promoter to effect transcription, an optional operator sequence to control transcription, a sequence encoding a suitable mRNA ribosome binding site, and sequences that control termination of transcription and translation.

The term “host cell” refers to a cell capable of maintaining a vector either transiently or stably. Host cells of the invention include, but are not limited to, bacterial cells, yeast cells, insect cells, plant cells and mammalian cells. Other host cells known in the art, or which become known, are also suitable for use in the invention.

The term “array” refers to a collection of elements, wherein each element is uniquely identifiable. For example, the term can refer to a substrate bearing an arrangement of elements, such that each element has a physical location on the surface of the substrate that is distinct from the location of every other element. In such an array, each element can be identifiable simply by virtue of its location. Typical arrays of this type include elements arranged linearly or in a two-dimensional matrix, although the term “array” encompasses any configuration of elements and includes elements arranged on non-planar, as well as planar, surfaces. Non-planar arrays can be made, for example, by arranging beads, pins, or fibers to form an array. The term “array” also encompasses collections of elements that do not have a fixed relationship to one another. For example, a collection of beads in which each bead has an identifying characteristic can constitute an array.

The elements of an array are termed “target elements.”

As used herein with reference to target elements, the term “distinct location” means that each element is physically separated from every other target element such that a signal (e.g., a fluorescent signal) from a labeled molecule bound to target element can be uniquely attributed to binding at that target element.

A “microarray” is an array in which the density of the target elements on a substrate surface is at least about 100/cm².

The term “active agent” is used herein to refer to any agent that elicits a biological response in vivo or in vitro, e.g., when added to cell or tissue culture.

A “normal” cell or tissue is one that is free of a particular physiological disorder of interest. Thus, a normal cell can be “abnormal” in some respect, but still be a “normal” cell for the purposes of the invention.

A “diseased” cell or tissue is one that is afflicted with a particular physiological disorder of interest.

In a mixture containing double-stranded and single-stranded polynucleotides, the single-stranded polynucleotides are said to be “functionally isolated” from the double-stranded polynucleotides if the single-stranded polynucleotides can be used preferentially as a template for synthesis of additional polynucleotide strands. For example, single-stranded polynucleotides can be functionally isolated from double-stranded polynucleotides by preferentially digesting the double-stranded polynucleotides with an enzyme.

II. General Subtractive Hybridization Method

A. In General

The invention provides a general subtractive hybridization method that facilitates the identification of one or more polynucleotides in a test polynucleotide sample that are absent from, or less abundant in, a reference polynucleotide sample. This method entails a first subtractive hybridization reaction to block high-abundance test polynucleotide sequences, leaving test polynucleotide strands that are enriched for low-abundance sequences. In a second subtractive hybridization reaction, low-abundance enriched polynucleotide sequences that are present in both samples are removed and test-specific duplexes are produced.

More specifically, the method uses driver polynucleotide strands that are substantially enriched in high-abundance polynucleotide sequences relative to either the test or the reference sample. The method entails contacting the high-abundance enriched driver polynucleotide strands with polynucleotide strands of, or prepared from, the test sample under hybridization conditions to form a first hybridization mixture, thereby blocking high-abundance polynucleotide sequences. This reaction produces unhybridized polynucleotide strands from the test sample that are enriched in low-abundance polynucleotide sequences relative to the test sample.

The unhybridized test polynucleotide strands then serve as a template for synthesizing low-abundance enriched test polynucleotide strands. The latter are then contacted under hybridization conditions with driver consisting of reference polynucleotide strands that are enriched in low-abundance polynucleotide sequences relative the reference sample. This second hybridization produces hybrid duplexes corresponding to sequences shared by both samples and unhybridized low-abundance enriched polynucleotide strands corresponding to sequences that differ between the samples. The hybrid duplexes are removed or digested to functionally isolate the unhybridized low-abundance enriched polynucleotide strands, and those corresponding to the test sample are rendered double-stranded. The resulting duplexes are low-abundance polynucleotide sequences that are absent from or substantially reduced in the reference sample. In this manner, test sample-specific or differentially expressed genes can be identified, even though their corresponding transcripts are present at low abundance in the test sample. An exemplary protocol for this method is given in Example 4.

As noted above, the driver for the first hybridization reaction can be derived from the test or reference sample. The method will be described herein with reference to preferred embodiments in which this driver consists of high-abundance enriched test polynucleotide strands. In particularly preferred embodiments, the high-abundance enriched test polynucleotide strands are high-abundance enriched antisense polynucleotide strands, which hybridize to sense test polynucleotide strands in the first hybridization mixture. However, those of skill in the art understand that the sense of the polynucleotide components of such hybridization reactions can be reversed, so that, for example, high-abundance enriched sense test polynucleotide strands are employed as driver in a hybridization with antisense test polynucleotide strands.

Where the high-abundance enriched polynucleotide strands are antisense polynucleotide strands, hybridization with sense test polynucleotide strands produces unhybridized sense test polynucleotide strands that are enriched in low-abundance polynucleotide sequences relative to the test polynucleotide sample. These unhybridized sense test polynucleotide strands then serve as a template for synthesizing low-abundance enriched antisense test polynucleotide strands. The latter are then contacted under hybridization conditions with sense reference polynucleotide strands that are enriched in low-abundance polynucleotide sequences relative the reference sample. The resulting hybrid duplexes are removed or digested to functionally isolate the unhybridized low-abundance enriched antisense polynucleotides, and those corresponding to the test sample are rendered double-stranded, preferably by selective amplification. This step produces low-abundance, “test-specific” duplexes that are generally cloned for further analysis.

Starting polynucleotides useful in the general subtractive hybridization method of the invention can be obtained from any source. In particular, DNA or RNA useful in the invention can be extracted and/or amplified from any source, including bacteria, yeast, viruses, organelles, as well higher organisms such as plants or animals, with mammals being preferred, and humans being most preferred. Starting polynucleotides can also be extracted or amplified from cells, bodily fluids (e.g., blood), or tissue samples by a variety of standard techniques. Starting polynucleotides useful in the invention can also be derived from polynucleotide libraries, including cDNA, cosmid, YAC, or BAC libraries, and the like; however, the use of such libraries is not preferred because, as discussed above, many of the low-abundance sequences of interest may not be present in such libraries.

The starting polynucleotides need not be in pure form, but must be sufficiently pure to allow the hybridization and synthetic reactions of the subtractive hybridization method to be performed.

In preferred embodiments, the test and reference polynucleotide strands are obtained from different polynucleotide samples. For example, the test and reference polynucleotide samples can be derived from: a first cell or tissue and a second, different cell or tissue; a cell or tissue at a first stage of differentiation or development and the same type of cell or tissue at a second stage of differentiation or development; a cell or tissue treated with an active agent and a cell or tissue that is untreated or treated with a second, different active agent; a normal cell or tissue and a diseased cell or tissue. Preferred examples of the latter include, but are not limited to, various diseases, such as infectious disease, inflammatory disease, cardiovascular discease, cancer, and/or a heritable disease.

Although the subtractive hybridization method can be applied to any type of polynucleotide sample, mRNA samples are preferably employed to study differences in gene expression between two samples (i.e., “expression monitoring”). As those of skill in the art appreciate, mRNA samples can be converted to cDNA, which can optionally be cloned to produce polynucleotide libraries. Similarly, amplified DNA representations of the mRNA present in a cell can be produced for use in the invention. As discussed above, however, such manipulations can result in the loss of polynucleotide sequences, especially low-abundance sequences, and thus, for this additional reason, the use of mRNA samples is preferred.

B. First Subtractive Hybridization to Block High-Abundance Polynucleotides

In the first subtractive hybridization reaction in the general hybridization method of the invention, a high-abundance enriched driver is hybridized to test polynucleotide strands to block high-abundance polynucleotide sequences. The driver generally consists of high-abundance enriched polynucleotide strands of, or prepared from, a pool of test or reference polynucleotides that is enriched in high-abundance polynucleotide sequences relative to the test or reference polynucleotide sample, respectively. Where high-abundance enriched reference polynucleotide strands are employed as driver, the first subtractive hybridization reaction blocks those polynucleotide sequences that are highly represented in the test and reference polynucleotide samples. Where high-abundance enriched test polynucleotide strands are employed as driver, the first subtractive hybridization reaction blocks those polynucleotide sequences that are highly represented in the test polynucleotide sample (not just those that are also highly represented in the reference polynucleotide sample). Preferably, the driver is produced from the sample that has the highest concentration of high-abundance sequences that are present in the test sample, which in many cases, is the test sample itself.

In preferred embodiments, the high-abundance enriched polynucleotide strands are antisense polynucleotide strands. Antisense polynucleotide strands for use in the subtractive hybridization method of the invention can be prepared from a polynucleotide sample by any means known to those of skill in the art. For example, antisense polynucleotide strands enriched in high-abundance polynucleotide sequences, relative to the polynucleotide sample from which they were derived, can be produced by taking advantage of the differences in reassociation kinetics between high-abundance and low-abundance sequences. If polynucleotides are denatured and allowed to reassociate, the sequences present in the sample at a higher copy number will reassociate before the lower-copy number sequences. Thus, sequences that become double-stranded relatively quickly (e.g., Cot=5.5 or less, where Co is moles of nucleotide/liter and t is time in seconds) represent high-abundance polynucleotide sequences. These double-stranded sequences can be recovered from the reassociation mixture and used to produce antisense polynucleotides for use in the general subtractive hybridization method of the invention. In a preferred embodiment, high-abundance enriched antisense polynucleotide strands are produced as described below in the section entitled “Methods for Preparing Selected Polynucleotide Pools.”

For subtractive hybridization, the high-abundance enriched polynucleotide strands are contacted with test polynucleotide strands under conditions wherein at least some of the polynucleotides specifically hybridize to one another. In preferred embodiments, high-abundance enriched antisense polynucleotide strands are contacted with sense test polynucleotide strands. Preferably, the antisense polynucleotide strands are antisense RNA molecules, and the sense test polynucleotide strands are mRNA molecules.

The antisense polynucleotide strands are usually added to the hybridization reaction in excess to drive hybridization (and are thus termed the “driver”), although this is not a requirement of the method. For most applications, the molar ratio of antisense polynucleotide to other polynucleotides in the reaction mixture is between about 1:1 and about 800:1, preferably between about 1:1 and about 200:1, and more preferably between about 1:1 and about 100:1, although other ratios are possible.

The hybridization reaction is carried out at high temperature, usually between about 60-70° C., to achieve relatively specific hybridization. In addition, buffers and salt concentrations used can be adjusted to achieve the necessary stringency using techniques known to those of skill in the art. Typically, fairly high stringencies are preferred. Accepted methods for conducting hybridization assays are known, and general overviews of the technology is found in: Nucleic Acid Hybridization: A Practical Approach, Ed. Hames, B. D. and Higgins, S. J., IRL Press, 1985; Hybridization of Nucleic Acids Immobilized on Solid Supports, Meinkoth, J. and Wahl, G.; Analytical Biochemistry, 238:267-284, 1984. Subtractive hybridization techniques are also specifically described in U.S. Pat. No. 5,589,339 (issued December, 31, 1996 to Hampson et al.), U.S. Pat. No. 5,935,788 (issued Aug. 10, 1999 to Burmer et al.), and U.S. Pat. No. 5,958,738 (issued Sep. 28, 1999 to Lindemann et al.).

If the high-abundance enriched polynucleotide strands contain, e.g., poly-U or poly-T sequences and the test polynucleotide strands contain, e.g., poly-A sequences, or vice versa, all of the high-abundance enriched polynucleotide strands would be expected to hybridize to all of the test polynucleotide strands and no subtraction would occur. To prevent this type of “non-specific” hybridization, blocking oligonucleotides (i.e., poly-A, poly-U, and/or poly-T) can be included in the hybridization mixture, usually in excess. Preferably, blocking oligonucleotides are employed at a molar ratio of between about 1:1 and about 800:1 blocking oligonucleotides:polynucleotides in the hybridization mixture. More preferably the ratio of blocking oligonucleotides to other polynucleotides is between about 1:1 and about 200:1, and even more preferably between about 1:1 and about 100:1, although other ratios are possible. Alternatively, the polynucleotide strands employed in the subtraction hybridization mixture can either be produced so that the strands do not contain sequences that would cause non-specific hybridization or such sequences can be removed by partial digestion with a suitable enzyme. For example, partial digestion with E. coli exonuclease I can be employed to remove poly-A tails from the 3′ ends of cDNAs.

Hybridization produces double-stranded polynucleotides corresponding to high-abundance polynucleotide sequences and unhybridized test polynucleotide strands that are enriched in low-abundance polynucleotide sequences relative to the test polynucleotide sample. If the high-abundance enriched polynucleotide strands employed for hybridization were derived from a reference, rather than a test, sample, the reaction mixture will generally also contain unhybridized high-abundance enriched reference polynucleotide strands.

C. Production of Low-Abundance Enriched Test Polynucleotide Strands

The next step of the general subtractive hybridization method of the invention is to use the unhybridized low-abundance enriched test polynucleotide strands produced in the first hybridization reaction as a template to produce complementary low-abundance enriched test polynucleotide strands.

1. Synthesis of Low-Abundance Enriched Test Polynucleotide Strands

In preferred embodiments, the unhybridized low-abundance enriched test polynucleotide strands are sense strands that serve as a template to synthesize antisense polynucleotide test strands that are likewise enriched in low-abundance polynucleotide sequences relative to original the test polynucleotide sample. Although any standard technique can be employed for this purpose, in a particularly preferred embodiment, synthesis of the antisense test polynucleotide strands is primed using an antisense primer.

The antisense primer hybridizes with an antisense primer site present in the sense test polynucleotide strands. This antisense primer site can be located at the 3′ end of the sense polynucleotide strands (e.g., the poly-A tail of mRNA molecules), which produces full-length antisense polynucleotide strands. Alternatively, the antisense primer site can be located so that antisense test polynucleotide strands are synthesized for only a portion of the sense test polynucleotide strands. In an example of the latter embodiment, random primers prime at random sites in the polynucleotide strands, which typically results internal priming to produce truncated polynucleotide strands.

Synthesis of antisense test polynucleotide strands is preferably primed using an antisense primer or, optionally, an antisense primer complex. The antisense primer complex has two components: (1) an antisense primer and (2) a specifically oriented RNA polymerase promoter sequence. An antisense primer complex is desirable if, for example, to facilitate the production of antisense RNA from the test-specific duplexes obtained using the general subtractive hybridization method of the invention.

The antisense primer is capable of acting as a point of initiation of polynucleotide synthesis, typically DNA replication, when placed under conditions suitable for primer extension, i.e., in the presence of appropriate nucleotides and a replicating agent (e.g., a DNA polymerase) under suitable reaction conditions, which are well known in the art. The primer is preferably a single-stranded oligonucleotide, most preferably an oligodeoxynucleotide. The primer must be sufficiently long and have a sequence that allows formation of a sufficiently stable duplex with the sense test polynucleotide strands to permit the synthesis of extension products in the presence of the replicating agent. The exact lengths of the primers and the quantities used will depend on many factors, including hybridization temperature, ionic conditions, degree of homology, and other considerations familiar to those of skill in the art. A primer designed to hybridize to a specific sequence motif typically contains between about 10 and about 50 nucleotides, and preferably between about 15 and about 25 or more nucleotides, although the primer can contain fewer nucleotides, depending, e.g., on the sequence motif. For other applications, the oligonucleotide primer is typically, but not necessarily, shorter, e.g., about 7 to about 15 nucleotides. As those of skill in the art readily appreciate, such short primer molecules generally require lower hybridization temperatures to form sufficiently stable hybrid complexes with template polynucleotides.

Antisense primers can be produced by any available method. Oligonucleotide primers are conveniently synthesized, for example, by the well-known phosphotriester and phosphodiester methods, especially the automated versions thereof. A standard automated method uses diethylphosphoramidites as starting materials, which can be purchased commercially or synthesized as described by Beaucage et al., Tetrahedron Letters 22: 1859-1962 (1981) or in U.S. Pat. No. 4,458,066. It is also possible to use primers that have been isolated from a biological source (e.g., via a restriction endonuclease digest or amplification).

Antisense primers useful in the methods of the invention are substantially complementary to the antisense primer sites in the sense polynucleotide strands. Therefore, a given antisense primer sequence need not be the exact complement of the antisense primer site to which it hybridizes. Non-complementary bases or longer sequences can be present the primer, provided that the primer sequence has sufficient complementarity with the sequence of the antisense primer site to permit hybridization and polynucleotide extension.

As stated above, in a preferred embodiment, the antisense primer hybridizes to low-abundance enriched sense test polynucleotide strands, such as mRNA molecules. In this case, the antisense primer can conveniently include a poly-T (also termed “oligonucleotide-dT” or “oligo-dT”) sequence. (This sequence generally includes about 5 to about 50, preferably about 5 to about 20, more preferably about 10 to about 15 T residues, which will hybridize with the poly-A tail present at the 3′ terminus of each unhybridized mRNA molecule. Alternatively, if only RNA sharing a common nucleotide sequence motif is to be amplified, then the antisense primer is substantially complementary to this sequence motif.

The second component of the antisense primer complex is an RNA promoter sequence. Such sequences are capable of binding an RNA polymerase and contain a transcriptional start site. The RNA promoter sequence employed in the antisense primer complex may be single-stranded or double-stranded. The promotor sequence usually includes between about 15 and about 250 nucleotides, preferably between about 25 and about 60 nucleotides, from a naturally occurring RNA polymerase promoter, a consensus promoter sequence (Alberts et al., in Molecular Biology of the Cell, 2d Ed., Garland, N.Y. (1989), or a modified version thereof.

A wide variety of promoters and polymerases showing specificity for their cognate promoter are known. In general, prokaryotic promoters are preferred over eukaryotic promoters, and phage or virus promoters are most preferred. Particularly preferred are the T3, T7, and SP6 phage promoter/polymerase systems. Probably the best studied is E. coli phage T7. T7 makes an entirely new polymerase that is highly specific for the 17 late T7 promoters. Rather than having two separate highly conserved regions like E. coli promoters, the late T7 promoters have a single highly conserved sequence from −17 to +6, relative to the RNA start site. The Salmonella phage SP6 is very similar to T7. Although most RNA polymerases recognize double-stranded promoters, E. coli phage N4 makes an RNA polymerase that recognizes early N4 promoters on native single stranded N4 DNA. A detailed description of promoters and RNA synthesis upon DNA templates is found in Watson et al., Molecular Biology of The Gene, 4th Ed., Chapters 13-15, Benjamin/Cummings Publishing Co., Menlo Park, Calif. A preferred promoter sequence is the sequence from the T7 phage that corresponds to its RNA polymerase binding site (5′-AAT TCT AAT ACG ACT CAC TAT AGG G-3′; SEQ ID NO:1).

The RNA promoter sequence is linked to the antisense primer to facilitate transcription in the presence of ribonucleotides and an RNA polymerase under suitable conditions. The primer and promoter components are linked with the RNA promoter upstream (5′) of the antisense primer in an orientation that permits transcription of a polynucleotide strand that is complementary to the primer, i.e., such that antisense RNA transcription (described in detail below) will generally be in the same direction as the primer extension. Any type of linkage that meets this criterion can be employed, however nucleotide linkages are preferred. A linker oligonucleotide between the components, if present, typically includes between about 5 and about 20 bases, but may be smaller or larger as desired.

In addition, the antisense primer or antisense primer complex, preferably includes at least one (first) restriction site, and more preferably including at least two restriction sites. The first restriction site is typically 5′ of the sequence in the antisense primer that binds to the template and 3′ of an additional sequence in the antisense primer that can serve as a primer site for amplification. Accordingly, the first restiction site can be used in a novel selective restriction cleavage method (described below) whereby hybrid duplexes are rendered incapable of amplification by restriction digestion to remove the amplification primer site, whereas single-stranded polynucleotides are left intact. In this embodiment, the restriction site is one that is cleaved by a restriction enzyme that cuts double-stranded DNA, but not single-stranded DNA. Examples include: Alul, BbvI, DpnI, FnuDII, FokI, HpaII, HphI, MboI, MboII, MspI, Sau3AI, SfaNI, and the like.

The second restriction site, if present, is generally 5′ of the first. The second restriction site can be used to facilitate cloning of the test-specific duplexes produced by the general subtractive hybridization method of the invention. This site is therefore preferably recognized by restriction enzymes that cut DNA relatively infrequently to reduce the likelihood of unwanted internal cleavage within the test-specific duplexes to be cloned. Such enzymes are well-known in the art. As illustrated in Example 4, the second restriction site can also serve as the amplification primer site. In this example, the antisense primer includes (3′ to 5′) a poly-T sequence, which hybridizes to poly-A tails of mRNA, linked to an Alu I site (first restriction site), which is linked to a Sfi I site (second restriction site).

Once the antisense primer, optionally with the operably linked promoter region, hybridizes to the sense test polynucleotides, antisense test polynucleotide strands are synthesized. If the sense polynucleotides are mRNA, a first strand of cDNA is conveniently produced through the process of reverse transcription, wherein DNA is made from RNA, utilizing reverse transcriptase according to standard techniques. This enzyme, which is present in all retroviruses (e.g., avian myeloblastoma virus), adds deoxyribonucleotides to the 3′ terminus of the primer (Varmus, Science 240: 1427-1435 (1988)). Reverse transcription produces antisense cDNA strands as the low-abundance enriched test polynucleotide strands.

2. Isolation of Low-Abundance Enriched Test Polynucleotide Strands

Immediately after synthesis of low-abundance enriched test polynucleotide strands, the reaction mixture includes, in addition, the template polynucleotide strands. It is generally desirable to separate the newly synthesized polynucleotide strands from the template so that the newly synthesized strands can be used in the second subtractive hybridization reaction of the general subtractive hybridization method of the invention.

Preferably, the newly synthesized strands are antisense strands, more preferably antisense cDNA strands, and the template preferably consists of sense polynucleotide strands, more preferably mRNA. RNA can be selectively degraded by any of a variety of conventional methods, leaving DNA in the mixture intact. As illustrated in the examples, treatment of the mixture with sodium hydroxide is one convenient method for removing RNA. Sodium hydroxide treatment is carried out under conditions such that DNA is not substantially degraded and can be used for subtractive hybridization. Other means of isolating low-abundance enriched test polynucleotide strands for this purpose can readily be devised by those of skill in the art for different applications of the general subtractive hybridization method of the invention.

D. Subtractive Hybridization to Block Low-Abundance Polynucleotides Present in the Test and Reference Samples

The newly synthesized low-abundance enriched test polynucleotide strands are then employed in a second subtractive hybridization reaction. The second subtractive hybridization reaction also includes low-abundance enriched polynucleotide strands of, or prepared from, a reference pool of polynucleotides that is enriched in low-abundance polynucleotide sequences relative to the reference polynucleotide sample. The low-abundance enriched reference polynucleotide strands serve as driver, which block low-abundance polynucleotides present in both samples.

Where the newly synthesized polynucleotide strands are antisense test polynucleotide strands, the low-abundance enriched reference polynucleotide strands are sense reference polynucleotide strands. Low-abundance enriched sense reference polynucleotide strands can be prepared from a polynucleotide sample by any means known to those of skill in the art. For example, sense polynucleotide strands enriched in low-abundance polynucleotide sequences, relative to the polynucleotide sample from which they were derived, can be produced by taking advantage of the differences in reassociation kinetics between high-abundance and low-abundance sequences, as described above. Sequences that become double-stranded relatively quickly (e.g., Cot=5.5 or less, where Co is moles of nucleotide/liter and t is time in seconds) represent high-abundance polynucleotide sequences. These double-stranded sequences can be removed from the reassociation mixture by methods known in the art (e.g., hydroxyapatite binding), leaving low-abundance enriched polynucleotides, which can used to produce corresponding sense polynucleotides for use in subtractive hybridization. In a preferred embodiment, low-abundance enriched antisense polynucleotide strands are produced as described below in the section entitled “Methods for Preparing Selected Polynucleotide Pools.”

For subtractive hybridization, the low-abundance enriched reference polynucleotide strands are contacted with the low-abundance enriched test polynucleotide strands under conditions wherein at least some of the polynucleotides specifically hybridize to one another. In preferred embodiments, low-abundance enriched sense reference polynucleotide strands are contacted with low-abundance antisense test polynucleotide strands. In a particularly preferred embodiment, the sense reference polynucleotide strands are sense cDNA strands, and the antisense test polynucleotide strands are antisense test cDNA strands.

The low-abundance enriched reference polynucleotide strands are usually added to the hybridization reaction in excess to drive hybridization, although this is not a requirement of the method. For most applications, the molar ratio of low-abundance enriched reference polynucleotides to other polynucleotides in the reaction mixture is between about 1:1 and about 800:1, preferably between about 1:1 and about 200:1, and more preferably between about 1:1 and about 100:1, although other ratios are possible.

The hybridization reaction is carried out at high temperature, usually between about 60-70° C., to achieve relatively specific hybridization. As described above for the first subtractive hybridization reaction, buffers and salt concentrations used can be adjusted to achieve the necessary stringency using techniques known to those of skill in the art. Typically, fairly high stringencies are preferred.

If the low-abundance enriched test polynucleotide strands contain, e.g., poly-U or poly-T sequences and the low-abundance enriched reference polynucleotide strands contain, e.g., poly-A sequences, or vice versa, all of the high-abundance enriched polynucleotide strands would be expected to hybridize to all of the test polynucleotide strands and no subtraction would occur. If desired, blocking oligonucleotides can be added to the hybridization mixture to prevent, e.g., hybridization between poly-A sequences in one collection of polynucleotide strands from hybridizing with poly-U or poly-T sequences in the other collection of polynucleotide strands.

Alternatively, the sequences that would promote non-specific hybridization can be removed from at least one collection of polynucleotide strands. For example, in a preferred embodiment, the low-abundance enriched test polynucleotide strands are antisense cDNA strands, and the low-abundance enriched reference polynucleotide strands are sense cDNA strands. The latter contain a poly-A tail, which can be removed, for example, by treating the sense cDNA strands with a 3′exonuclease, under controlled conditions, to remove the poly-A tails. See Example 4, in which E. coli exonuclease I (available commercially from New England Biolabs) is used to remove poly-A tails from sense reference cDNA strands.

Finally, at least one collection of polynucleotide strands can be produced in a manner that eliminates sequences that would promote non-specific hybridization. For example, in a preferred embodiment, sense reference cDNA strands are produced from antisense reference RNA for subtractive hybridization with antisense test cDNA strands. If the antisense reference RNA contains a poly-U tail, the sense reference cDNA strand produced from this RNA would contain a corresponding poly-A stretch. Poly-U tails can be eliminated from antisense reference RNA by heating (e.g., at 72° C. for about 60 min. or at 90° C. for about 5 min.) to degrade the poly-U tails. This “poly-U-removed” antisense RNA is then used synthesize sense reference cDNA strands that lack a corresponding poly-A stretch.

Hybridization produces hybrid duplexes corresponding to low-abundance abundance polynucleotide sequences present in both of the test and reference samples, unhybridized low-abundance enriched test polynucleotide strands, and unhybridized low-abundance enriched reference polynucleotide strands.

E. Removal of Hybrid Duplexes

Because the general subtractive hybridization method of the invention is directed toward isolating test-specific polynucleotides, the hybrid duplexes representing common sequences are preferably removed or digested. Methods for separating double-stranded and single-stranded polynucleotides are well known, and any available method can be employed in the invention. Alternatively, double-stranded polynucleotide can be selectively degraded by nuclease digestion. Nucleases that selectively digest double-stranded polynucleotides are well known and include, for example, lambda nuclease, E. coli exonuclease III, and the like. Buffers, times, and temperatures suitable for digestion of double-stranded polynucleotides are known in the art.

In addition, as noted above, many restriction restriction enzymes cleave only double-stranded polynucleotides. Accordingly, the invention provides a novel selective restriction cleavage method that functionally isolates single-stranded polynucleotides in a mixture of single- and double-stranded polynucleotides. This method entails contacting the polynucleotide mixture with one or more restriction enzymes under conditions sufficient to allow digestion of double-stranded polynucleotides to a form that cannot serve as a template for nucleotide synthesis. In one embodiment, a restriction enzyme, preferably a frequent cutter, such as those with four-base long recognition sites, can be employed to cut double-stranded polynucleotide into smaller fragments. This cleavage, in effect, functionally isolates the single-stranded polynucleotides, allowing selective synthesis of polynucleotide products corresponding to the single-stranded polynucleotides. The reaction conditions, such as buffers, times, and temperatures, for digestion of double-stranded polynucleotides are well known for the various restriction enzymes suitable for use in the invention.

In a preferred embodiment, the low-abundance enriched test polynucleotide strands and/or the low-abundance enriched reference polynucleotide strands employed in the hybridization reaction each have a restriction site near the 5′ terminus. When one of each of these polynucleotide strands hybridizes, a restriction site can be produced at one or both ends of the resulting hybrid duplex.

Typically, in this embodiment, the hybrid duplexes formed when low-abundance enriched test polynucleotide strands hybridize with low-abundance enriched reference polynucleotide strands are not double-stranded at the 5′ restriction sites. Accordingly, a standard “fill in” reaction is carried out to render the molecules double-stranded at these sites. Enzymes useful for this fill-in reaction do not differ from those used for fill-in reactions in other contexts. For example, Taq DNA polymerase is conveniently employed to fill in 5′ overhangs, as shown in Example 4. Buffers and reaction conditions suitable for such enzymes are well known.

Where the low-abundance enriched test polynucleotide strands are antisense test polynucleotide strands, the restriction site can be incorporated into the antisense primer or antisense primer complex used to synthesize the antisense test polynucleotide strands. As discussed above, the primer or primer complex can be designed so that the restriction site is 3′ of a sequence that serves as a downstream primer site in a subsequent amplification reaction. In this embodiment, cleavage at the restriction site in double-stranded polynucleotides separates the primer site from the remainder of the polynucleotide. This cleavage converts the double-stranded polynucleotide to a form that cannot serve as a template for amplification. Thus, restriction enzyme digestion functionally isolates the single-stranded polynucleotides, allowing selective nucleotide synthesis, e.g., amplification, of the antisense test polynucleotide strands.

An analogous restriction site can be incorporated into low-abundance enriched sense reference polynucleotide strands when these strands are produced as described below in the section entitled “Preparation of a Polynucleotide Pool that is Enriched in Low-Abundance Polynucleotide Sequences.” This restriction site is also 3′ of a sequence that serves as an upstream primer site for amplification. Accordingly, cleavage at this site also separates the primer site from the remainder of the polynucleotide, thereby preventing amplification.

In an alternative embodiment, 3′ universal primer site(s) can be added to both ends of the molecules in the hybridization reaction mixture after the fill-in reaction and prior to restriction enzyme digestion. See Example 4. The universal primer site is typically present in an oligonucleotide that is ligated or otherwise linked the test polynucleotide strands. The oligonucleotide can be an oligodeoxynucleotide, an oligoribodeoxynucleotide, or a hybrid molecule containing deoxynucleotides and ribodeoxynucleotides. The universal primer site should have a length and sequence suitable for hybridizing to a universal primer. In preferred embodiments, the universal primer site serves as an “anchor” for an amplification reaction, which is conveniently carried out using the polymerase chain reaction (“PCR”). The considerations for selecting a suitable universal primer site sequence are well known in the art. The usual and preferred lengths for such sequences are the same as those given above for the antisense primer.

The universal primer site can be added to the 3′ ends of polynucleotide strands by any convenient method, such as, for example, “oligonucleotide-tailing” or ligation.

In oligonucleotide-tailing (also termed “homopolymeric tailing”), deoxynucleotides of a particular type, i.e., dA, dT, dG, or dC, are added to the 3′ end of a DNA strand using a terminal transferase. This reaction produces a DNA strand with an oligonucleotide-dA, -dT, -dG, or -dC tail that can serve as a universal primer site for an oligonucleotide-dT, -dA, -dC, or -dG primer, respectively.

The universal primer site can also be added by ligating an oligonucleotide to the 3′ end of the antisense polynucleotide strand, as described, for example in Akowitz, Gene 81:295-306 (1989).

In a preferred embodiment, after the fill-in reaction, oligonucleotide-tailing is be employed to add an oligonucleotide tail, which can serve as a primer site for an appropriate homopolymeric primer. Subsequent digestion with a suitable restriction enzyme removes the oligonucleotide tail from hybrid duplexes, but not from single-stranded polynucleotides, which can then serve as a template for amplification.

F. Production of Test-Specific Duplexes from Unhybridized Low-Abundance Enriched Test Polynucleotide Strands

The removal or digestion of hybrid duplexes produced in the second subtractive hybridization reaction leaves substantially intact low-abundance enriched test and reference polynucleotide strands. The test and reference polynucleotide strands have opposite senses. In a preferred embodiment, the reference polynucleotide strands are sense reference polynucleotide strands, and the test polynucleotide strands are antisense test polynucleotide strands.

The method entails selectively rendering the low-abundance enriched test polynucleotide strands double-stranded, thereby producing low-abundance enriched test-specific duplexes. The selective conversion of low-abundance enriched test polynucleotide strands to test-specific duplexes is illustrated herein with reference to the embodiment in which the low-abundance enriched test polynucleotide strands are antisense test polynucleotide strands that have a 5′ antisense primer site and that have a 3′ universal primer site. Preferably, the low-abundance enriched reference polynucleotide strands employed in the second hybridization do not have these elements or, if such elements are present, their nucleotide sequences are sufficiently different from those in the antisense test polynucleotide strands that the antisense test polynucleotide strands can be selectively amplified. Alternatively, in a preferred embodiment described in Example 4, the reference polynucleotide strands contain a 5′ primer site and a 3′ primer site that can self-anneal, which prevents amplification. In example 4, a 5′ poly-G sequence anneals with a 3′ poly-C sequence. Other strategies that allow selective conversion of low-abundance enriched test polynucleotide strands to test-specific duplexes can be designed by those of skill in the art in light of the guidance herein and are therefore within the scope of the invention.

In one embodiment, the antisense test polynucleotide strands are rendered double-stranded using an amplification reaction. Amplification is preferably carried out by PCR, and more preferably by enhanced PCR, both of which are well known to those of skill in the art. PCR is described in U.S. Pat. Nos. 4,683,202, 4,683,195, 4,800,159, and 4,965,188, as well as in Saiki, Science 230:1350 (1985). PCR entails hybridizing two primers to substantially complementary sequences that flank a target sequence in a polynucleotide. A repetitive series of reaction steps involving template denaturation, primer annealing, and extension of the annealed primers by a DNA polymerase results in the geometric accumulation of a the target sequence whose termini are defined by the 5′ ends of the primers. As denaturation is typically carried out at temperatures that denature most DNA polymerases (e.g., about 93° C.-95° C.), a thermostable polymerase, such as those derived from Thermus thermophilus, Thermus aquaticus (Taq), or Thermus flavus, is typically used for extension to avoid the need to add additional polymerase for each extension cycle.

In a preferred embodiment, antisense test polynucleotide strands are amplified using enhanced PCR. Enhanced PCR can be carried out as described, for example, in U.S. Pat. No. 5,436,149 (issued Jul. 25, 1995 to Barnes et al.), which discloses the use of a polymerase combination including a variant of Taq or Thermus flavus DNA polymerase lacking 3′-exonuclease activity and a lesser amount of a thermostable DNA polymerase having such activity. A similar polymerase combination that can also be used in the method is described in U.S. Pat. No. 5,512,462 (issued Apr. 30, 1996 to Cheng). The considerations affecting the selection of PCR primers and amplification conditions are well known, and those of skill in the art can readily determine primers and conditions suitable for a particular application of the method of the invention.

PCR amplification of antisense test polynucleotide strands that have a 5′ antisense primer site and a 3′universal primer site is conveniently accomplished, for example, by using a universal primer that hybridizes to the universal primer site as the 5′primer and using the antisense primer or antisense primer complex as the 3′ primer. Thus, such primers are preferably selected to serve as anchors for efficient and sufficiently specific PCR amplification.

In a preferred embodiment illustrated in Example 4, the antisense test polynucleotide strands are antisense cDNA strands contain that a 5′ poly-T sequence and a poly-C tail added by oligonucleotide tailing in the selective restriction cleavage method described above. The hybridization reaction mixture also contains sense reference polynucleotide strands that contain the poly-C tail, but not the poly-T sequence. The antisense test polynucleotide strands can thus be selectively amplified using a poly-G primer as the 5′ primer and a poly-A primer as the 3′ primer.

In a particularly preferred variation of this embodiment, restriction sites that facilitate cloning of the test-specific duplexes are introduced during the amplification. Thus, as described in Example 4, the 5′ primer includes a restriction site, such as Not I, linked to the 5′ end of the poly-G primer sequence. Instead of using a poly-A primer as the 3′ primer, a primer containing an Sfi I site is used. The Sfi I primer hybridizes to the Sfi I site at the 5′ end of the antisense test polynucleotide strands. Amplification thus produces duplexes with 5′ Not I sites and 3′ Sfi I sites, which can be used to clone the test-specific duplexes into a vector of choice. As described above, it is generally desirable to introduce restriction sites for enzymes whose sites appear infrequently in DNA to reduce cutting within the test-specific duplexes. As those of skill in the art readily appreciate, test duplexes having the same restriction site at either end can also be produced, although test duplexes with different restriction sites at either end are preferred for directional cloning.

After amplification, the test-specific duplexes produced from the antisense test polynucleotide strands, are typically cloned into a vector using standard recombinant DNA techniques, which are described below in the section entitled “Uses of Polynucleotide Pools.” This cloning step represents a simple means of isolating the test-specific sequences from all other sequences in the amplification reaction mixture. The resulting polynucleotide library represents a “subtracted polynucleotide pool,” which contains low-abundance polynucleotide sequences that are not present in the reference polynucleotide sample or are present in the reference polynucleotide sample in substantially lower concentration than in the test polynucleotide sample.

III. Methods for Preparing Selected Polynucleotide Pools

The invention also provides methods for selecting polynucleotide pools from a polynucleotide sample. These polynucleotide pools can be used to produce driver for the first and second subtractive hybridization reactions described above. Other applications for such pools are described in detail below.

One selection method exploits the loss of low-abundance sequences during dilution to produce a polynucleotide pool that is enriched in high-abundance polynucleotide sequences relative to the polynucleotide sample. This method can be employed, for example, to obtain a pool of test or reference polynucleotides that is enriched in high-abundance polynucleotide sequences relative to a test or reference polynucleotide sample, respectively. Such a pool can be employed as driver in the first subtractive hybridization reaction described above.

A second selection method begins with a high-abundance polynucleotide pool produced by the dilution method of the invention or otherwise. This method relies on subtractive hybridization with the high-abundance polynucleotide pool to produce a low-abundance-enriched polynucleotide pool. This selection method can be employed to obtain a pool of reference polynucleotide strands that is enriched in low-abundance polynucleotide sequences relative to a reference polynucleotide sample. A low-abundance enriched reference polynucleotide pool can be used, for example, as driver in the second subtractive hybridization reaction described above.

Both of the methods for preparing selected polynucleotide pools are described in application Ser. No. 09/632,898 (filed Aug. 7, 2000), which is explicitly incorporated by reference herein.

A. Preparation of a Polynucleotide Pool that is Enriched in High-Abundance Polynucleotide Sequences

1. Synthesis of First Antisense Polynucleotide Strands from a Polynucleotide Sample

To prepare a polynucleotide pool that is enriched in high-abundance polynucleotide sequences, first antisense polynucleotide strands can be synthesized from sense polynucleotide strands of a polynucleotide sample or sense polynucleotide strands prepared from a polynucleotide sample.

Essentially any polynucleotides can be used as the starting polynucleotides for the preparation of a high-abundance enriched polynucleotide pool, provided they each contain nucleotide sequences substantially complementary to an antisense primer. This antisense primer site can be located at the 3′ end of the sense polynucleotide strands (e.g., the poly-A tail of mRNA molecules), which produces full-length antisense polynucleotide strands. Alternatively, the antisense primer site can be located so that antisense polynucleotide strands are synthesized for only a portion of the sense polynucleotide strands (e.g., as when a random primer mixture is used).

Starting polynucleotides useful in the selection methods of the invention can be obtained from any source, as described above for those useful in the general subtractive hybridization method. The starting polynucleotides need not be present initially in a pure form; they can be a minor fraction of a complex mixture, provided that other components in the mixture do not substantially interfere with the synthesis of the first antisense polynucleotide strands.

In preferred embodiments, synthesis of first antisense polynucleotide strands is primed using an antisense primer complex. As stated above, the antisense primer complex includes: (1) an antisense primer and (2) a specifically oriented RNA polymerase promoter sequence. The antisense primer complex can also contain a sequence that is a restriction endonuclease site (restriction site), which can facilitate selective restriction cleavage (see above) or cloning of polynucleotide pools produced according to the methods of the invention.

The considerations for an antisense primer useful in the selection methods of the invention are the same as discussed above with respect to antisense primers useful in the general subtractive hybridization method of the invention. Briefly, the primer is preferably a single-stranded oligonucleotide, most preferably an oligodeoxynucleotide. A primer designed to hybridize to a specific sequence motif typically contains between about 10 and about 50 nucleotides, and preferably between about 15 and about 25 or more nucleotides, although the primer can contain fewer nucleotides, depending, e.g., on the sequence motif. For other applications, the oligonucleotide primer is typically, but not necessarily, shorter, e.g., about 7 to about 15 nucleotides.

The second component of the antisense primer complex is an RNA promoter sequence. RNA promoter sequences useful in the selection methods of the invention are as described above with respect to the general subtractive hybridization method. Accordingly, the promotor sequence usually includes between about 15 and about 250 nucleotides, preferably between about 25 and about 60 nucleotides, from a naturally occurring RNA polymerase promoter, a consensus promoter sequence (Alberts et al., in Molecular Biology of the Cell, 2d Ed., Garland, N.Y. (1989), or a modified version thereof. Particularly preferred for use in the selection methods of the invention are the T3, T7, and SP6 phage promoter/polymerase systems.

The RNA promoter sequence is linked to the antisense primer to facilitate transcription in the presence of ribonucleotides and an RNA polymerase under suitable conditions. The primer and promoter components are linked with the RNA promoter upstream (5′) of the antisense primer in an orientation that permits transcription of a polynucleotide strand that is complementary to the primer, i.e., such that antisense RNA transcription will generally be in the same direction as the primer extension. Any type of linkage that meets this criterion can be employed, however nucleotide linkages are preferred. A linker oligonucleotide between the components, if present, typically includes between about 5 and about 20 bases, but may be smaller or larger as desired.

In a preferred embodiment, a selected polynucleotide pool is produced from RNA. Total RNA can be employed, but poly-A RNA (i.e., mRNA) is preferable for most applications. In either case, a plurality of antisense primer complexes containing antisense primers of random sequence (i.e., “random primers”) can be employed. To produce a selected polynucleotide pool from the mRNA present in a sample, the antisense primer can include an oligo-dT sequence (e.g. about 5 to about 50, preferably about 5 to about 20, more preferably about 10 to about 15 T residues. If only RNA sharing a common nucleotide sequence motif is to be amplified, then the primer is substantially complementary to this sequence motif. When preparing a high-abundance enriched polynucleotide pool for use as driver in the first subtractive hybridization reaction discussed above, the starting polynucleotides are preferably mRNA, and the antisense primer complex preferably comprises random primers.

After hybridization of the antisense primer and operably linked promoter region to the sense polynucleotides in a sample, first antisense polynucleotide strands are synthesized. If the sense polynucleotides are mRNA, first antisense cDNA strands are conveniently produced through the process of reverse transcription, as described above.

2. Addition of a Universal Primer Site

Synthesis of first antisense polynucleotide strands from the sense polynucleotide strands of, or prepared from, the polynucleotide sample, is preferably followed by the addition of a universal primer site to the 3′ ends of the first antisense polynucleotide strands, as described above. The universal primer site should have a length and sequence suitable for hybridizing to a universal primer, and is preferably designed to serve as an anchor for an amplification reaction, such as PCR. The universal primer site can optionally include a restriction site, to facilitate selective restriction cleavage or cloning of polynucleotide pools of the invention. The universal primer site can be added to the first antisense polynucleotide strands by any convenient method, including oligonucleotide-tailing, and ligation, which are described above, as well as by “template switching.”

If the sense polynucleotide strands are mRNA and the first antisense strands are cDNA, template switching is carried out as follows. A template-switching oligonucleotide is included during reverse transcription, which produces an mRNA-antisense cDNA hybrid. The template-switching oligonucleotide hybridizes to the CAP site at the 5′end of mRNA strand and serves as a short, extended template for CAP-dependent extension of the 3′ end of the antisense cDNA strand. Template-switching oligonucleotides typically require a few ribonucleotides at the 3′ end to promote CAP-dependent extension. Thus, template-switching oligonucleotides generally contain between about 1 and about 5 ribonucleotides at their 3′ ends.

3. Dilution of First Antisense Polynucleotide Strands

After production of first antisense polynucleotide strands, preferably containing universal primer sites, the reaction mixture is diluted to substantially eliminate at least some low-abundance antisense polynucleotide strands. Serial dilution is typically employed for this purpose, and the degree of dilution depends upon the desired abundance threshold. Minimal dilution removes the rarest polynucleotide stands in the mixture, whereas greater dilution removes polynucleotide strands that are present in higher copy number. Dilutions useful for standard applications of the method range from about 10⁻¹, about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, and about 10⁻¹², although higher or lower dilutions may be desirable in specific applications. A serial dilution is made by removing an aliquot of the reaction mixture and transferring the aliquot to a volume of an aqueous solution that provides the desired degree of dilution. If desired, multiple transfers may be used to achieve a stepwise dilution that yields the desired degree of dilution. The aqueous solution used for dilution is preferably one that is compatible the enzymes used in the next step of the method to produce double-stranded polynucleotides from the first antisense polynucleotide strands present after dilution.

4. Production of First Double-Stranded Polynucleotides from Remaining First Antisense Polynucleotide Strands

First double-stranded polynucleotides can be produced from the first antisense polynucleotide strands remaining after dilution by any of a number of available methods. Where the first antisense polynucleotide strands are first antisense cDNA strands, second-strand cDNA can be synthesized using RNase H and E. coli DNA polymerase, optionally including DNA ligase. RNase assists in breaking the RNA/first-strand cDNA hybrid, and DNA polymerase synthesizes a complementary DNA strand using the first-strand cDNA as template. The second strand is generated as deoxynucleotides are added to the 3′ terminus of the growing strand. If the first antisense cDNA strands include an RNA promoter sequence at the 5′ end, the single-stranded promoter sequence is copied into the double-stranded promoter region in the desired orientation.

In a preferred embodiment, first double-stranded polynucleotides are produced by amplification. If the first antisense polynucleotide strands are cDNA molecules in a RNA/first-strand cDNA hybrid, amplification can be carried out under conditions such that the RNA is degraded. Alternatively, the RNA can be removed prior to amplification by any suitable technique, such as, for example, treatment with sodium hydroxide. Amplification is preferably carried out by PCR, and more preferably by enhanced PCR. PCR amplification of first antisense polynucleotides that have a 3′universal primer site is conveniently accomplished, for example, by using a universal primer that hybridizes to the universal primer site as the 5′primer and using the antisense primer complex as the 3′ primer. Thus, such primers are preferably selected to serve as anchors for efficient and sufficiently specific PCR amplification.

Where the first antisense polynucleotide strands were generating using an antisense primer complex mixture comprising random primers, a random sequence linked to an RNA promoter sequence is incorporated into the antisense polynucleotide strands. In this embodiment, the RNA promoter sequence can serve as primer site for amplification. Accordingly, the amplification reaction would include a 3′ primer that binds to the RNA promoter sequence at the 5′ end of each antisense polynucleotide strand.

The pool of polynucleotides produced from first antisense polynucleotide strands is enriched in high-abundance polynucleotide sequences with respect to the starting polynucleotide sample and can be used, for example, in a subtractive hybridization reaction to produce a selected polynucleotide pool that is enriched in low-abundance polynucleotide sequences relative to the starting polynucleotide sample, as described in greater detail below. For clarity, the high-abundance-enriched polynucleotides are termed the “first double-stranded polynucleotides,” and the low-abundance-enriched polynucleotides discussed below are termed the “second double-stranded polynucleotides.”

Where the polynucleotide sample is an mRNA sample, the first double-stranded polynucleotides are referred to as “cDNA” molecules. In preferred embodiments, the first double-stranded polynucleotides retain the universal primer site at the 5′ end (relative to the sense strand) and a functional RNA polymerase at the 3′ end (relative to the sense strand).

5. Synthesis of Antisense RNA from First Double-Stranded Polynucleotides

Antisense RNA can be synthesized from first double-stranded polynucleotides containing an RNA promoter by contacting the polynucleotides with an RNA polymerase capable of binding to the RNA promoter region under conditions suitable for RNA synthesis. The sense strand is transcribed into antisense RNA. Amplification occurs because the polymerase repeatedly recycles on the template (i.e., reinitiates transcription from the promoter region). This technique permits the replication of a broad range of polynucleotides without the need for cloning into vectors. In addition, recycling of the polymerase on the same template avoids propagation of errors.

The RNA polymerase used for the transcription must be capable of operably binding to the particular promoter region employed in the antisense primer complex described above. Substantially any polymerase/promoter combination can be used; however, bacteriophage RNA polymerases, in particular from T3, T7, and SP6 phages, are preferred. The most preferred polymerase is T7 RNA polymerase. The extremely high degree of specificity shown by T7 RNA polymerase for its promoter site (Chamberlin et al., in The Enzymes, ed. P. Boyer (Academic Press, New York) pp. 87-108 (1982)) has previously made this enzyme a useful reagent in a variety of recombinant DNA techniques, including in vitro RNA synthesis from plasmids containing the promoter site for use as probes (Melton et al., Nucl. Acids Res., 12: 7035-7056 (1984)), for in vitro translation studies (Krieg et al., Nuc. Acids Res. 12: 7057-7070 (1984)), and for use in producing synthetic oligoribonucleotides (Milligan et al., Nuc. Acids Res. 15: 8783-8798 (1987)). The lack of efficient termination signals for T7 polymerase also enables this enzyme to transcribe almost any DNA sequence (see, Rosenberg et al., Gene 56: 125-135 (1987)). Finally, T7 polymerase is available from a number of commercial sources, such as Promega Biotech, Madison, Wis., and in a concentrated form (1000 units/·1) from Epicenter Technologies, Madison, Wis. E. coli RNA polymerase can also be employed with an appropriate E. coli RNA polymerase promoter region.

The transcription reaction mixture includes the necessary nucleotide triphosphates, which may be modified, depending on the ultimate use of the antisense RNA. For example, if the antisense RNA is intended for use as a nucleic hybridization probe, one or more of the nucleotides may be labeled, as described in greater detail below.

B. Preparation of a Polynucleotide Pool that is Enriched in Low-Abundance Polynucleotide Sequences

1. Subtractive Hybridization of a Polynucleotide Sample with Antisense Polynucleotide Strands that are Enriched in High-Abundance Polynucleotide Sequences

Antisense polynucleotide strands that are enriched in high-abundance polynucleotide sequences can be prepared from a polynucleotide sample by any convenient method, including those described above with respect to the general subtractive hybridization method of the invention. In a preferred embodiment, the high-abundance enriched polynucleotide strands are antisense RNA transcribed from first double-stranded polynucleotides, as described above. The antisense RNA is hybridized with sense polynucleotides of, or prepared from, the polynucleotide sample. This hybridization reaction produces double-stranded polynucleotides and unhybridized sense polynucleotides. Because the antisense RNA is enriched in high-abundance sequences, the high-abundance sequences become double-stranded, and the unhybridized sense polynucleotides are enriched in low-abundance sequences relative to the polynucleotide sample.

The antisense polynucleotide strands and the sense polynucleotide strands used in subtractive hybridization are preferably derived from the same polynucleotide sample. Thus, to produce a reference pool of polynucleotides enriched in low-abundance sequences for use in the second hybridization reaction of the general subtractive hybridization method described above, the antisense reference polynucleotide strands are preferably hybridized with sense reference polynucleotide strands. However, the selection method of the invention also encompasses the subtractive hybridization of antisense polynucleotide strands derived from one polynucleotide sample and sense polynucleotide strands of, or prepared from, a different polynucleotide sample. In this case, subtractive hybridization would block high-abundance sequences shared by the two samples.

The antisense polynucleotide strands are usually added to the hybridization reaction in excess to drive hybridization although this is not a requirement of the method. For most applications, the molar ratio of antisense polynucleotide to other polynucleotides in the reaction mixture is between about 1:1 and about 800:1, preferably between about 1:1 and about 200:1, and more preferably between about 1:1 and about 100:1, although other ratios are possible. The subtractive hybridization is carried out as described above for the first and second subtractive hybridization reactions of the general subtractive hybridization method of the invention.

In a preferred embodiment, the antisense polynucleotide strands are antisense RNA molecules of or prepared from a pool of high-abundance enriched polynucleotides (e.g., high-abundance enriched reference polynucleotides), and the sense polynucleotide strands are mRNA molecules from the same sample. In this case, subtractive hybridization produces unhybridized mRNA molecules that are enriched in low-abundance polynucleotide sequences, relative to the sample, as the unhybridized sense polynucleotide stands.

2. Synthesis of Second Antisense Polynucleotide Strands from Unhybridized Sense Polynucleotide Strands

The unhybridized sense polynucleotide strands from the subtractive hybridization reaction can then, if desired, be used as templates for the synthesis of another set of antisense polynucleotide strands. Although any standard technique can be employed for this purpose, in preferred embodiments, this second set of antisense polynucleotide strands is synthesized using an antisense primer or an antisense primer complex, as described above. If an antisense primer complex is employed, the antisense polynucleotide strands contain a primer site and an RNA promoter sequence at the 5′ end. Thus, an antisense primer complex is preferably employed if it is desirable to produce a pool of selected polynucleotides that each include an RNA promoter to facilitate the synthesis of antisense RNA from the selected polynucleotides. If the primer site and/or the RNA promoter sequence are to be used to initiate nucleotide synthesis in reaction mixtures that may contain undesired polynucleotides also having primer sites and/or promoter sequences, the primer sites and/or promoter sequences are preferably sufficiently different to allow specific nucleotide synthesis from the desired polynucleotides. (See, e.g., Example 4, in which the primer and RNA promoter sequences employed in B.2. are different from those employed in A.1. and the primer site is different from that employed in C.2.) Regardless of the method employed, the second antisense polynucleotide strands produced from the unhybridized sense polynucleotides are enriched in low-abundance sequences relative to the polynucleotide sample.

Where the unhybridized sense polynucleotide are mRNA molecules, the mRNA is conveniently reverse transcribed to produce second antisense cDNA strands as the second antisense polynucleotide strands.

3. Addition of a Universal Primer Site

In preferred embodiments of the invention, a universal primer site is added to the 3′ end of the second antisense polynucleotide strands as described above to facilitate simultaneous synthesis and amplification of second double-stranded polynucleotides that are enriched in low-abundance sequences. The universal primer site is preferably added by template switching, oligonucleotide-tailing, or ligation. If the primer site is to be used to initiate nucleotide synthesis in reaction mixtures that may contain undesired polynucleotides also having a universal primer site, the primer site incorporated into the second antisense polynucleotide strands is preferably sufficiently different to allow specific nucleotide synthesis from the desired polynucleotides. (See, e.g., Example 4, in which the universal primer site employed in B.3. is different from that employed in A.2.)

4. Production of Second Double-Stranded Polynucleotides from Second Antisense Polynucleotide Strands

Second double-stranded polynucleotides can be produced from second antisense polynucleotide strands as described above. If a 3′ universal primer site has been added to the antisense polynucleotide stands, the polynucleotide strands are preferably amplified using PCR, and more preferably using enhanced PCR. This amplification is conveniently carried out using a universal primer that hybridizes to the universal primer site as the 5′ primer and using the antisense primer or antisense primer complex as the 3′ primer. These primers can include a restriction site, if desired, to facilitate selective restriction cleavage or cloning of polynucleotide pools of the invention. The resultant pool of polynucleotides is enriched in low-abundance polynucleotide sequences with respect to the starting polynucleotide sample.

If the second antisense polynucleotide strands are cDNA strands in a RNA/first-strand cDNA hybrid, the RNA sequences are preferably removed prior to amplification by any suitable technique, such as, for example, treatment with sodium hydroxide. Amplification then produces double-stranded cDNA molecules.

IV. Polynucleotide Pools

A. In general

The invention provides polynucleotide pools that can be prepared using the methods of the invention. Such pools include a plurality of different polynucleotide sequences, generally including at least about 10², at least about 10², at least about 10⁴, at least about 10⁵, at least about 10⁶, or at least about 10⁷ different polynucleotide sequences. In preferred embodiments, the polynucleotides each include an RNA promoter sequence and a universal primer site. The polynucleotides can be any form of DNA or RNA and can be single- or double-stranded. Preferably, the polynucleotides are double- or single-stranded cDNA or anti sense RNA.

In one embodiment, the invention provides a subtracted polynucleotide pool prepared by subtractive hybridization between test and reference polynucleotide samples, as in the general subtractive hybridization method described above. Generally, the substracted polynucleotide pool comprises a library of polynucleotide clones. The subtracted polynucleotide pool is substantially enriched in polynucleotide sequences that are not present in the reference polynucleotide sample or that are present in the reference polynucleotide sample in substantially lower concentration than in the test polynucleotide sample. In preferred embodiments, the enrichment is on the order of about 10³-, about 10⁴-, about 10⁵-, about 10⁶-, or about 10⁷-fold. The subtracted polynucleotide pool is also substantially enriched in low-abundance polynucleotide sequences, relative to the test polynucleotide sample. Preferably, the low-abundance polynucleotides are about 10³-, about 10⁴-, about 10⁵-, about 10⁶-, or about 10⁷-fold enriched. Subtracted polynucleotide pools according to the invention are typically produced as libraries of polynucleotides cloned into a vector.

Selected polynucleotide pools resulting from the selection methods of the invention contain a plurality of polynucleotides prepared from a polynucleotide sample that is substantially enriched in high- or low-abundance polynucleotide sequences relative to the polynucleotide sample. Selected polynucleotide pools can comprise a library of polynucleotide clones or a pool of uncloned polynucleotides. In preferred embodiments, the high- or low-abundance polynucleotide sequences are about 10³-, about 10⁴-, about 10⁵-, about 10⁶-, or about 10⁷-fold enriched, relative to the polynucleotide nucleotide sample.

The polynucleotide pools of the invention are useful in a wide variety of applications. Although the following description discusses uses of the pools, those of skill in the art understand that an individual polynucleotide can be selected from a polynucleotide pool and used essentially as described for the pools.

B. Uses of Polynucleotide Pools

1. Nucleotide Synthesis

Polynucleotide pools prepared according to the invention can also be used as templates for cDNA synthesis and/or subjected to amplification to further expand one or more desired sequences. Amplification is preferably carried out by PCR, and more preferably by enhanced PCR. The entire pool can be amplified, preferably using an appropriate antisense primer or antisense primer complex and universal primer, or a subset of the pool can be amplified. Individual sequences of interest can be amplified using at least one, and preferably two, gene-specific primers. Alternatively, antisense RNA can be synthesized from the polynucleotide pools as described above.

2. Hybridization

The polynucleotide pools, or polynucleotides (such as antisense RNA) produced from them, can be used in a hybridization reaction. If desired, the pools or polynucleotides produced therefrom can be labeled with a detectable label. A wide variety of labeling techniques are well known to those skilled in the art and can be used to produce labeled polynucleotides of the invention in accordance with standard procedures (see U.S. Pat. No. 4,755,619). The labeling step can be incorporated into one of the above-described reactions so that the above-described methods produce labeled polynucleotide pools. For example, one or more nucleotide triphosphates can be included in a reaction mixture. Suitable labels are well known and include, for example, a radioactive label, such as ³²S, ³²P, ³H, and the like, or a non-radioactive label, such a fluorescent label. Labeling may be direct or indirect. In an example of the latter, one or more biotinylated nucleotides is used to synthesize biotinylated polynucleotides (see, Sive and St. John, Nucl. Acids Res. 16: 10937 (1988) and Duguid et al., Proc. Natl. Acad. Sci. USA 85: 5738-5742 (1988)). The biotinylated polynucleotides can then be detected by binding to labeled avidin.

3. Driver in Subtractive Hybridization

In another application, the polynucleotide pools of the invention are particularly useful for producing polynucleotides intended for use as driver in subtractive hybridization protocols. Such protocols typically require large amounts (generally tens of micrograms) of driver. This requirement makes it difficult to examine differential expression of mRNAs present in a biological material that is available in small supply. This difficulty has been addressed by cloning the polynucleotide collections of interest prior to subtraction, so that the cloning vector is used to amplify the amount of polynucleotide available for hybridization. However, because subtraction requires previous cloning, it is complicated, suffers from under- and over-representation of sequences depending on differences in growth rates in the mixed population, and may risk recombination among sequences during propagation of the mixed population.

The methods of the present invention circumvent these problems by allowing production of large amounts of antisense RNA from limited amounts of polynucleotides, without the need for previous cloning. These methods are superior to PCR, which produces both sense- and antisense strands that must be separated before use in subtractive hybridization. High- or low-abundance antisense RNA produced as described above can be used in methods of detecting and isolating polynucleotides that vary in abundance among different populations, for example, allowing mRNA expression to be compared among different tissues or within the same tissue according to physiologic state.

In a preferred embodiment, antisense RNA synthesized from a pool of high-abundance enriched polynucleotides derived from a test or reference mRNA sample is employed in the first subtractive hybridization reaction of the general subtractive hybridization method of the invention. The antisense RNA blocks the high-abundance polynucleotide sequences in the test mRNA, leaving low-abundance enriched mRNA strands free to serve as a template for nucleotide synthesis.

In another preferred embodiment, antisense RNA synthesized from a pool of low-abundance enriched polynucleotides derived from a reference mRNA sample is employed in the second subtractive hybridization reaction of the general subtractive hybridization method. More specifically, the low-abundance enriched antisense reference RNA is used as a template to synthesize sense low-abundance enriched sense reference cDNA strands. Example 4 illustrates a preferred embodiment of this application, in which cDNA synthesis is primed using a universal primer including a restriction site at its 5′ end. The cDNA strand synthesized has a 5′ restriction site, a universal primer site, a poly-A sequence, and a 3′promoter sequence. The latter two elements are preferably removed by partial digestion with an exonuclease, such as E. coli exonuclease I. The resultant low-abundance enriched sense reference cDNA strands lack poly-A sequences that would hybridize “non-specifically” with poly-T-containing molecules. These sense reference cDNA strand are conveniently employed as driver in a subtractive hybridization reaction to block the low-abundance polynucleotide sequences that are present in both the reference and test polynucleotide samples, leaving low-abundance enriched test polynucleotide strands that are absent from, or less abundant in, the reference sample. See Example 4. The unblocked low-abundance enriched test polynucleotide strands can then serve as a template for nucleotide synthesis to produce double-stranded polynucleotides.

4. Nucleotide Arrays

The polynucleotide pools of the invention, or polynucleotides produced therefrom, can, if desired, be attached to one or more substrates to produce a polynucleotide array, which can then be used in a hybridization assay. In a preferred embodiment, each type of polynucleotide constitutes a different target element in the array. Preferably, a polynucleotide pool of the invention is used to produce a DNA microarray.

Arrays of polynucleotides of the invention can be produced in accordance with conventional techniques for DNA array fabrication. For example, a sample dispenser mounted on a device that can be precisely positioned can be employed to spot samples onto a substrate. U.S. Pat. No. 5,807,522 (issued Sep. 15, 1998 to Brown and Shalon) describes a device that facilitates mass fabrication of microarrays characterized by a large number of micro-sized assay regions separated by a distance of 50-200 microns or less and a well-defined amount of analyte (typically in the picomolar range) associated with each region of the array.

An alternative approach to robotic spotting uses an array of pins or capillary dispensers dipped into the wells, e.g., the 96 wells of a microtiter plate, for transferring an array of samples to a substrate. Arrays can also be fabricated by coating elements such as beads or optical fibers with samples to form target elements. U.S. Pat. No. 5,830,645 (issued Nov. 3, 1998 to Pinkel et al.) describes the use of beads to produce a polynucleotide array, and U.S. Pat. No. 5,690,894 (issued on Nov. 25, 1997 to Pinkel et al.) discloses a polynucleotide array fabricated from optical fibers.

5. Cloning

Polynucleotide pools prepared according to the above methods can be cloned into vectors using standard cloning techniques to produce polynucleotide libraries. Such libraries can facilitate studies of gene expression in essentially any cell or cell population. The subject cells may be obtained from blood (e.g., white cells, such as T or B cells) or other tissues, such as brain, spleen, bone, heart, vascular, lung, kidney, liver, pituitary, endocrine glands, lymph nodes, dispersed primary cells, tumor cells, and the like. In the area of neural research, for example, the identification of mRNAs that vary as a function of, e.g., arousal state, behavior, drug treatment, and development has been hindered by both the difficulty of constructing cDNA libraries from small brain nuclei. Use of polynucleotide pools in accordance with the invention to construct cDNA libraries from individual brain nuclei provides for greater representation of low-abundance mRNAs from these tissues compared with their representation in whole-brain cDNA libraries and facilitates the cloning of important low-abundance messages.

Vectors suitable for use in cloning typically contain a replication sequence capable of effecting replication of the vector in a suitable host cell (i.e., an origin of replication) as well as sequences encoding a selectable marker, such as an antibiotic resistance gene. Upon introduction of the vector into a suitable host, the vector can replicate and function independently of the host genome or integrate into the host genome. Vector design depends, among other things, on the intended use and host cell for the vector, and the design of a vector of the invention for a particular use and host cell is within the level of skill in the art.

In a preferred embodiment, the polynucleotides of the invention encode polypeptides and are cloned into expression vectors. Expression vectors include one or more control sequences capable of effecting and/or enhancing the expression of an operably linked protein coding sequence. Control sequences that are suitable for expression in prokaryotes, for example, include a promoter sequence, an operator sequence, and a ribosome binding site. Control sequences for expression in eukaryotic cells include a promoter, an enhancer, and a transcription termination sequence (i.e., a polyadenylation signal). An expression vector useful in the methods of the invention can also include other sequences, such as, for example, sequences encoding a signal sequence or an amplifiable gene. A signal sequence directs the secretion of a polypeptide fused thereto from a cell expressing the protein. The inclusion in a vector of a gene complementing an auxotrophic deficiency in the chosen host cell allows for the selection of host cells transformed with the vector.

A vector of the present invention is typically produced by linking desired elements by ligation at convenient restriction sites. Cloning can be simplified if the antisense primer or antisense primer complex and the universal primer site used to generate the polynucleotides of the invention include restriction sites. The inclusion of different restriction sites in the primer or primer complex and the universal primer site facilitates directional cloning. Preferably, the restriction sites used occur infrequently in the polynucleotides of the original sample, to minimize internal cutting of the polynucleotides of the invention. Examples of suitable sites include those recognized by SfiI and NotI.

Vectors containing the cloned polynucleotides can be introduced into host cells. A wide variety of host cells are available for propagation and/or expression of vectors. Examples include prokaryotic cells (such as E. coli and strains of Bacillus, Pseudomonas, and other bacteria), yeast or other fungal cells (including S. cerevesiae and P. pastoris), insect cells, and plant cells, as well as higher eukaryotic cells (such as human embryonic kidney cells and other mammalian cells). Host cells according to the invention include cells in culture and cells present in live organisms, such as transgenic plants or animals.

Vectors can be introduced into host cells by any convenient method, which will vary depending on the vector-host system employed. Generally, a vector is introduced into a host cell by transformation (also known as “transfection”) or infection with a virus (e.g., phage) bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by infection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, and protoplast fusion also are acceptable for use in the invention.

6. Expression of Encoded Polypeptides

Host cells transformed with expression vectors can be used to express the polypeptides encoded by the cloned polynucleotides of the invention. Expression entails culturing the host cells under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well-known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium or a cell lysate by any method capable of separating the polypeptide from one or more components of the host cell or culture medium. Typically, the polypeptide is separated from host cell and/or culture medium components that would interfere with the intended use of the polypeptide. As a first step, the culture medium or cell lysate is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an affinity domain, purification typically includes the use of an affinity column containing the cognate binding partner. For instance, polypeptides fused with hexahistidine or similar metal affinity tags can be purified by fractionation on an immobilized metal affinity column.

7. Other Research and Therapeutic Uses

Polynucleotide pools of the invention also have a wide variety of uses in both research and therapeutics. Antisense RNA, for example, functions in several prokaryotic systems to regulate gene expression. Similarly, antisense RNA can regulate the expression of many eukaryotic genes. This permits blocking the expression of undesirable genes or of unknown genes in studies aimed at identifying their function.

Research or therapeutic use of polynucleotides of the invention can, for example, involve in vitro production of the polynucleotides, with subsequent introduction into cells or a subject (see, generally, Melton, Antisense RNA and DNA, Cold Spring Harbor (1988). The polynucleotides can be double-stranded or single-stranded, and single-stranded polynucleotides can be sense or antisense. Fragments of polynucleotides can also be employed. The use of antisense RNA oligonucleotides to inhibit transcription and thereby block gene expression is well known, and those of skill in the art can readily design antisense oligonucleotides based on the polynucleotides of the invention. See, e.g., Reeder et al. Cancer Res. 58:3719-26 (1998). In addition, single-stranded sense and double-stranded polynucleotides can also be employed to block gene expression by RNA interference (also termed “cosuppression” or “quelling”), which entails targeted mRNA degradation. See, e.g., Fire et al., Nature 391:806-811 (1998); Sharp and Zamore, Science 287:2431 (2000); and Marx, Science 288:1370 (2000).

For research or therapeutic applications, the polynucleotides are typically combined with a physiologically acceptable carrier, exipient, and/or stabilizer and/or a component that facilitates entry of the polynucleotide into a cell. Physiologically acceptable carriers, excipients, and stabilizers are described in Remington's Pharmaceutical Sciences (1980) 16th editions, Osol, ed., 1980. A physiologically acceptable carrier, excipient, or stabilizer suitable for use in the invention is non-toxic to cells or subjects at the dosages employed and can include a buffer (such as a phosphate buffer, citrate buffer, and buffers made from other organic acids), an antioxidant (e.g., ascorbic acid), low-molecular weight (less than about 10 residues) polypeptide, a protein (such as serum albumin, gelatin, and an immunoglobulin), a hydrophilic polymer (such as polyvinylpyrrolidone), an amino acid (such as glycine, glutamine, asparagine, arginine, and lysine), a monosaccharide, a disaccharide, and other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the physiologically acceptable carrier is an aqueous pH-buffered solution.

Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with the polynucleotides of the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC).

In another embodiment, liposomally entrapped polynucleotides can be employed to deliver the polynucleotides to cells. Liposomes can be composed of various types of lipids, phospholipids, and/or surfactants. These components are typically arranged in a bilayer formation, similar to the lipid arrangement of biological membranes. Liposomes containing polynucleotides are prepared by known methods, such as, for example, those described in Epstein, et al., PNAS USA 82:3688-92 (1985), and Hwang, et al., PNAS USA, 77:4030-34 (1980). Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Polynucleotides can also be complexed to dendrimers, which can be used to transfect cells. Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimes and various dendrimer polycations.

Dendrimer polycations are preferably non-covalently associated with the polynucleotides of the invention. This permits an easy disassociation or disassembling of the composition once it is delivered into the cell. Typical dendrimer polycations suitable for use herein have a molecular weight ranging from about 2,000 to 1,000,000 Da, and more preferably about 5,000 to 500,000 Da. However, other molecular weights can also be employed. Preferred dendrimer polycations have a hydrodynamic radius of about 11 to 60 Å.; and more preferably about 15 to 55 Å. Other sizes, however, are also suitable for use in the invention. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For prophylactic or therapeutic use, polynucleotides of the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides. In a preferred embodiment, prophylactic or therapeutic compositions of the invention include polynucleotides combined with lipids, as described above.

Compositions of the invention can be stored in any standard form, including, e.g., an aqueous solution or a lyophilized cake. Such compositions are typically sterile when administered to cells or recipients. Sterilization of an aqueous solution is readily accomplished by filtration through a sterile filtration membrane. If the composition is stored in lyophilized form, the composition can be filtered before or after lyophilization and reconstitution.

In some applications, it is advantageous to stabilize the polynucleotides described herein or to produce polynucleotides that are modified to better adapt them for particular applications. To this end, the polynucleotides of the invention can contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH₂—NH—O—CH₂, CH₂—N(CH₃)—O—CH₂ (known as the methylene(methylimino) or MMI backbone) and CH₂—O—N(CH₃)—CH₂, CH₂—N(CH₃)—N(CH₃)—CH₂, and O—N(CH₃)—CH₂—CH backbones (where phosphodiester is O—P—O—CH₂). Also preferred are polynucleotides having morpholino backbone structures. Summerton, J. E. and Weller, D. D., U.S. Pat. No. 5,034,506. Other preferred embodiments use a protein-nucleic acid or peptide-nucleic acid (PNA) backbone, wherein the phosphodiester backbone of the polynucleotide is replaced with a polyamide backbone, the bases being bound directly or indirectly to the aza nitrogen atoms of the polyamide backbone. P. E. Nielsen, M. Egholm, R. H. Berg, O. Buchardt, Science 1991, 254, 1497. Polynucleotides of the invention can contain alkyl and halogen-substituted sugar moieties and/or can have sugar mimetics such as cyclobutyls in place of the pentofuranosyl group. In other preferred embodiments, the polynucleotides can include at least one modified base form or “universal base” such as inosine. Polynucleotides can, if desired, include an RNA cleaving group, a cholesteryl group, a reporter group, an intercalator, a group for improving the pharmacokinetic properties of the polynucleotide, and/or a group for improving the pharmacodynamic properties of the polynucleotide.

V. Kits

The materials for use in the methods of the present invention are ideally suited for preparation of kits produced in accordance with well-known procedures. In one embodiment, a kit of the invention includes: (1) an antisense primer complex including an antisense primer linked to an RNA promoter sequence, wherein the RNA promoter sequence is 5′ of the antisense primer; (2) a sense primer; and (3) instructions for performing a method of the invention. While the instructional materials typically comprise written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media can include addresses to internet sites that provide such instructional materials. Preferred kits include one or more of the various reagents (typically in concentrated form) utilized in the methods, including, for example, one or more buffers, the appropriate nucleotide triphosphates (e.g., dATP, dCTP, dGTP and dTTP; or rATP, rCTP, rGTP and UTP), reverse transcriptase, DNA polymerase, and/or RNA polymerase.

In another embodiment, a kit of the invention includes: (1) a polynucleotide pool of the invention, (2) an antisense primer complex as described above, and (3) a sense primer. This kit is useful for preparing amplified DNA from the selected polynucleotide pool. Preferred kits include one or more containers, each with one or more reagents for amplifying DNA, e.g., a buffer, nucleotide triphosphates and/or a DNA polymerase.

In yet another embodiment, a kit includes: (1) a polynucleotide pool of the invention, and (2) an RNA polymerase capable of transcribing antisense RNA from the selected polynucleotide pool. Preferred kits include one or more containers, each with one or more reagents for producing antisense RNA, e.g., a buffer and/or nucleotide triphosphates.

All publications cited herein are explictly incorporated by reference.

### EXAMPLES

The following examples are offered to illustrate, but not to limit the claimed invention.

### Example 1

### Production of cDNA Enriched in Low-Abundance Sequences from Human Placental mRNA

A. Synthesis of First-Strand cDNA and PCR

1. Universal PCR Anchor Added by Template-Switching

First-strand cDNA was synthesized from human placental polyA+ RNA using a random primer linked to a T7 promoter sequence (Random-T7 primer): 5′-AAT-TCT-AAT-ACG-ACT-CAC-TAT-AGG-GNN-NN-NN-3′ (N=A, T, C or G; SEQ ID NO:2). Briefly, a 5-μl cDNA synthesis mixture containing 1 μl human placenta polyA+ RNA (1 μg), 1 μl Random-T7 primer (20 μl), 1 μl PCR anchor Oligo (also called Template-Switching Oligo: 5′-TGC-TGC-GAG-AAG-ACG-ACA-GAA-GGG-3′, (the 3′ “GGG” shown in bold were ribonucleotides; SEQ ID NO:3), and 2 μl of deionized H₂O. The mixture was incubated at 72° C. for 2 min and then 37° C. for 2 min. The volume was then adjusted to 10 μl with the following reagents: 2 μl of 5× first-strand synthesis buffer (250 mM Tris-HCl, pH 8.3; 30 mM MgCl₂; and 375 mM KCl), 0.5 μl of dithiothreitol (DTT; 50 mM), 1 μl of dNTP mix (10 mM each dATP, dCTP, dGTP, dTTP), 0.5 μl Rnasin (20 units, Promega), and 1 μl (200 units) of MMLV reverse transcriptase (SuperscriptII™, Life Technologies). The reverse transcription was carried out at 42° C. for 90 min. The reaction was terminated by placing the tube on ice.

First-strand cDNA was diluted to about 1 pg/μl (about 1×10⁻⁶) to eliminate rare transcripts, followed by PCR amplification in a 100 μl reaction containing 40 mM Tricine-KOH, pH 9.2; 15 mM KOAc; 3.5 mM Mg(OAc)₂; and 0.2 μM 5-′anchor primer (5′-TGC-TGC-GAG-AAG-ACG-ACA-GAA-3′); 0.2 μM Random-T7 primer; 0.2 mM each of dATP, dGTP, dCTP and dTTP; and 2 μl of Advantage™ cDNA Polymerase Mix (50×; contains KlenTaq-1 and Deep Vent polymerases; Clontech). PCR was carried out in a DNA Thermal Cycler 480 (PE Biosystems) using the following cycling conditions: 95° C. for 1 min; 30-35 cycles at 95° C. for 15 sec; 62° C. for 30 sec; and 72° C. for 6 min.

After PCR, double-stranded cDNA was purified by passing it over an S-200 spin column (Clontech). Before antisense RNA driver synthesis, the double-stranded cDNA was tested for amplification of glucose 3-phosphate dehydrogenase (G3PDH) and c-myc genes. After 25 cycles of PCR amplification, G3PDH was visible as an intense band on a 1.1% agarose gel stained with ethidium bromide. c-myc was not detectable on an ethidum bromide-stained gel, even after 35 cycles of amplification.

2. Universal PCR Anchor Added by Homopolymeric Tailing

First-strand cDNA was synthesized on human placenta polyA+ RNA using the Random-17 primer of Method 1. Briefly, a 5 μl cDNA synthesis mixture containing 1 μl human placental polyA+ RNA (1 μg), 1 μl Random-17 primer (20 μM), and 3 μl of deionized H₂O were incubated at 72° C. for 2 min and then 37° C. for 2 min. The volume was then adjusted to 10 μl with the following reagents: 2 μl of 5× first-strand synthesis buffer (250 mM Tris-HCl, pH 8.3; 30 mM MgCl₂; and 375 mM KCl), 0.5 μl of DTT (50 mM), 1 μl of dNTP mix (10 mM each dATP, dCTP, dGTP, dTTP), 0.5 μl Rnasin (20 units, Promega) and 1 μl (200 units) of MMLV reverse transcriptase (SuperscriptII™, Life Technologies). The reverse transcription was carried out at 42° C. for 90 min. The reaction was terminated by placing the tube on ice.

Homopolymeric tailing was carried out as previously reported (D. J. Bertioli et al., BioTechnology, 1994) with following modifications. 20 μl of deionized H₂O was added to the first-strand cDNA mixture. The diluted first-stand cDNA mixture was purified using a CHROMA SPIN-100 column (Clontech) to eliminate primers and dNTPs as well as small cDNA fragments. Oligo-dC tailing was carried out by mixing the following: 30 μl purified first-strand cDNA; 1 μl 5× tailing buffer (0.5 M potassium cacodylate, pH 7.2, 10 mM CoCl₂, 1 mM DTT), 1 μl of 1 mM dCTP, 1 μl terminal transferase (15 units/μl; Life Technologies), 8 μl deionized H₂O (final volume was 50 μl). The reaction mixture was incubated at 37° C. for 1 h, followed by phenol/chloroform extraction and precipitation.

The Oligo-dC-tailed cDNA was diluted to about 10 pg/μl (about 1×10⁻⁵) to eliminate rare transcripts, followed by PCR amplification in a 100 μl reaction containing 40 mM Tricine-KOH, pH 9.2; 15 mM KOAc; 3.5 mM Mg(OAc)₂; and 0.2 μM 5′-dG₁₂ primer (5′-d(G)₁₂-VN-3′, N=A, G, C, or T; V=A, G or C); 0.2 μM Random-T7 primer, 0.2 mM each of dATP, dGTP, dCTP and dTTP; and 2 μl of Advantage™ cDNA Polymerase Mix (50×; contains KlenTaq-1 and Deep Vent polymerases; Clontech). PCR was carried out in a DNA Thermal Cycler 480 (PE Biosystems) using following cycling conditions: 95° C. for 1 min.; 30-35 cycles at 95° C. for 15 sec; 52° C. for 30 sec; and 72° C. for 6 min.

After PCR, double-stranded cDNA was purified by passing passing it over an S-200 spin column (Clontech). Before antisense RNA driver synthesis, the double-stranded cDNA was tested for amplification of the (G3PDH) and c-myc genes. After 25 cycles of PCR amplification, G3PDH was visible as an intense band on a 1.1% agarose gel stained with ethidium bromide. c-myc was not detectable on an ethidum bromide-stained gel, even after 35 cycles of amplification.

B. Antisense RNA Synthesis

Antisense RNA was synthesized by mixing the following: the above double-stranded cDNA (1 μg); 5×RNA translation buffer (200 mM Tris-HCl, pH 8.5; 60 mM MgCl₂; 350 mM KCl; 25 mM DTT; 2.5 mM ITP; 7.5 mM GTP; 10 mM ATP; 10 mM U1P; 10 mM CIP), 40 units of RNasin (Promega), and 80 units of T7 RNA polymerase (Boehringer Mannheim) were then added. H₂O was added to a final reaction volume of 50 μl. The mixture was incubated at 41° C. for 90 min. 10 units of DNase I (RNase free; Boehringer Mannheim) were then added. This reaction was incubated at 37° C. for 15 min, followed by phenol/chloroform extraction and precipitation.

C. Subtractive Hybridization

Subtractive hybridization was performed using the above human placenta antisense RNA (enriched in high-abundance sequences) as a “driver” and normal human placenta mRNA (same origin as antisense RNA) as “tester.” Table 1 lists the various amounts of antisense RNA and placental mRNA in each reaction mixture.

TABLE 1
The Ratio of antisense RNA and mRNA for Subtractive Hybridization

| Sample | A | B | C | D |
|---|---|---|---|---|
| Placental | 1 μg (1 μl) | 1 μg (1 μl) | 1 μg (1 μl) | 1 μg (1 μl) |
| mRNA | | | | |
| Placental | Omitted | 0.5 μg (0.5 μl) | 1 μg (1 μl) | 2 μg (2 μl) |
| antisense RNA | | | | |
| DEPC H₂O | 2 μl | 1.5 μl | 1 μl | Omitted |
| Total | 3 μl | 3 μl | 3 μl | 3 μl |

After mixing, the tubes were incubated in a PE 9600 Thermal Cycler at 68° C. for 20 min. The temperature was then increased to 45° C. for a 16 h incubation.

D. cDNA Synthesis

Samples A, B, C, and D (above) were used as templates for first-strand cDNA synthesis using an Oligo-d(T)₃₀ primer (5′-d(T)₃O-VN-3′, N=A, G, C, or T; V=A, G or C). Briefly, 2 μl 10 mM Oligo-d (T)₃₀ was added to each sample, followed by incubation at 72° C. for 2 min. The reaction mixtures were then immediately transferred to an ice water bath for 2 min. The volume was adjusted to 10 μl with the following reagents: 2 μl of 5× first-strand synthesis buffer (250 mM Tris-HCl, pH 8.3; 30 mM MgCl₂, and 375 mM KCl), 0.5 μl of DTT (50 mM), 1 μl of dNTP mix (10 mM each dATP, dCTP, dGTP, dTTP), 0.5 μl Rnasin (20 units, Promega) and 1 μl (200 units) of MMLV reverse transcriptase (SuperscriptII™, Life Technologies). The reverse transcription was carried out at 42° C. for 90 min. The reactions were terminated by placing the tubes on ice.

E. Oligo-dG Tailing

After first-strand synthesis, RNA was degraded by adding 1 μl of 100 mM NaOH to each first-strand synthesis mixture and incubating at 68° C. for 30 min. 19 μl of deionized H₂O was then to each first-strand mixture, and DNA was purified using a Chroma Spin 100™ column (Clontech). Oligo-dG tailing was carried out by mixing the following: 30 μl purified first-strand cDNA; 1 μl 5× tailing buffer (0.5 M potassium cacodylate, pH 7.2, 10 mM CoCl₂, 1 mM DTT), 1 μl of 1 mM dGT?, 1 μl terminal transferase (15 units/μl; Life Technologies), 8 μl deionized H₂O (final volume was 50 μl). The reaction mixtures were incubated at 37° C. for 1 h, followed by phenol/chloroform extraction and precipitation.

F. Double-Stranded cDNA Synthesis by Low-Cycle PCR and Analysis of PCR Products 1. 0.9 kb Placental Transcript:

The subtraction of low-abundance sequences in samples A-D was assayed by examining the level of cDNA corresponding to a 0.9 kb placental “housekeeping” transcript. Low-cycle PCR amplification was carried out by dissolving the pellet obtained in the previous step in a 100 μl reaction containing 40 mM Tricinc-KOH, pH 9.2; 15 mM KOAc; 3.5 mM Mg(OAc)₂; and 0.2 μM 5′-dC₁₂ primer (5′-d(C)₁₂-VN-3′, N=A, G, C, or T; V=A, G or C); 0.2 μM Oligo-d(T)₃₀; 0.2 mM each of DATP, dGTP, dCTP and dTTP; and 2 μl of Advantage™ cDNA Polymerase Mix (50×; contains KlenTaq-1 and Deep Vent polymerases; Clontech). PCR was carried out in a DNA Thermal Cycler 480 (PE Biosystems) using following cycling conditions: 95° C. for 1 min; 8 cycles at 95° C. for 15 sec; 52° C. for 30 sec; and 72° C. for 6 min. PCR products from samples A, B, C and D were loaded onto a 1.1% agarose gel (5 μl/well), along with 200 ng 1 kb DNA ladder (Life Technologies). Staining with ethidium bromide revealed that the banding profile varied with the ratio of antisense mRNA driver to placental mRNA. Specifically, the intensity of a band at 0.9 kb corresponding to a human placental housekeeping gene decreased with increasing ratio of antisense RNA/mRNA, and was nearly undetectable in sample D, indicating that antisense RNA driver hybridizes to high-abundance mRNA sequences and subtracts them.

2. Glucose 3-Phosphate Dehydrogenase (G3PHD):

The subtraction of high-abundance sequences in samples A-D was also assayed by examining the level of cDNA corresponding to the housekeeping gene G3PHD. Samples A, B, C and D were each PCR-amplified in a 50 μl reaction mixture containing 40 mM Tricine-KOH, pH 9.2; 15 mM KOAc; 3.5 mM Mg(OAc)₂; and 0.2 μM 5′ and 3′G3PHD Amplimer set (Clontech); 0.2 mM each of DATP, dGTP, dCTP and dTTP; and 2 μl of Advantage™ cDNA Polymerase Mix (50×; contains KIenTaq-1 and Deep Vent polymerases; Clontech). PCR was carried out in a DNA Thermal Cycler 480 (PE Biosystems) using following cycling conditions: 95° C. for 1 min; 25 cycles at 95° C. for 15 sec; 68° C. for 2 min.

PCR products from samples A, B, C and D were loaded onto a 1.1% agarose gel (5 μl/well), along with 200 ng 1 kb DNA ladder (Life Technologies). Staining with ethidium bromide revealed that the intensity of the band corresponding to the housekeeping gene G3PHD decreased with increasing ratio of antisense RNA/mRNA and was nearly undetectable in sample D, confirming that antisense RNA driver subtracts high-abundance mRNA sequences.

3. c-myc:

The c-myc gene was used as a marker to assay the enrichment of low-abundance sequences in samples A-D. Each sample was PCR-amplified as described in the G3PHD study except that the 5′ and 3′ c-myc Amplimer set (Clontech) were used as primers, and PCR was carried out on using following cycling conditions: 95° C. for 1 min; then 30 cycles at 95° C. for 15 sec; and 68° C. for 2 min.

PCR products from samples A, B, C and D were loaded onto a 1.1% agarose gel (5 μl/well), along with 200 ng 1 kb DNA ladder (Life Technologies). Staining with ethidium bromide revealed that the intensity of c-myc amplicon increased with increasing ratio of antisense RNA/mRNA. The results indicate that c-myc was present in the normal human placental RNA, but not in the antisense RNA driver, and that subtractive hybridization, followed by amplification, produces a polynucleotide pool enriched in low-abundance sequences, such as c-myc.

### Example 2

### Production of cDNA Enriched in Low-Abundance Sequences from mRNA Prepared from Various Tissues

To test reproducibility of the methods of the invention, antisense RNA driver was produced from human brain, fetal brain, liver, and kidney mRNAs, as described in Example 1. An mRNA sample from each tissue was subjected to subtractive hybridization with antisense RNA driver prepared from the same tissue (group A). Negative controls (i.e., not treated with antisense RNA) were included for each sample (group B). After subtraction, low-cycle PCR was carried out to generate double-stranded cDNAs corresponding to unhybridized mRNA sequences.

The P50 gene was used as a marker to assay the enrichment of low-abundance sequences. Each sample was amplified in a 50 μl reaction containing 40 mM Tricine-KOH, pH 9.2; 15 mM KOAc; 3.5 mM Mg(OAc)₂; and 0.2 μM 5′ and 3′ P50 Amplimer set (Clontech); 0.2 mM each of dATP, dGTP, dCTP and dTIP; and 2 μl of Advantage™ cDNA Polymerase Mix (50×; contains KlenTaq-1 and Deep Vent polymerases; Clontech). PCR was carried out in a DNA Thermal Cycler 480 (PE Biosystems) using following cycling conditions: 95° C. for 1 min; 30 cycles at 95° C. for 15 sec; and 68° C. for 2 min.

Analysis of the PCR products by electrophoresis on a 1.1% agarose gel, followed by staining with ethidium bromide revealed that group A samples (enriched for low-abundance sequences) contained P50 sequences, whereas group B samples (negative control) contained no detectable P50 sequences. The results indicate that the methods of the invention are effective for producing selected polynucleotide pools, regardless of tissue type.

### Example 3

### Production of cDNA Enriched in Low-Abundance Sequences of Varying Length from mRNA Prepared from Human Testis mRNA

To test for enrichment of low-abundance sequences of varying length, antisense RNA driver was produced from human testis mRNAs, as described in Example 1. A human testis mRNA sample was subjected to subtractive hybridization with the antisense RNA driver (to generate a group A sample). A negative control (i.e., not treated with antisense RNA) was included (to generate a group B sample). After subtraction, low-cycle PCR was carried out to produce double-stranded cDNAs corresponding to unhybridized mRNA sequences.

Interleukin-6 (IL6, 0.6 kb ORF fragment), P50 (2.8 kb ORF), and Insulin Growth Factor Receptor (IGFR; 4.8 kb ORF fragment) were used as markers to check low-abundance gene enrichment and size representation. A and B samples were PCR-amplified in a 50 μl reaction containing 40 mM Tricine-KOH, pH 9.2; 15 mM KOAc; 3.5 mM Mg(OAc)₂; and 0.2 μM 5′ and 3′ IL6-, P50-, or IGFR-specific primer set; 0.2 mM each of dATP, dGTP, dCTP and dTTP; and 2 μl of Advantage™ cDNA Polymerase Mix (50×; contains KlenTaq-I and Deep Vent polymerases; Clontech). PCR was carried out in a DNA Thermal Cycler 480 (PE Biosystems) using following cycling conditions: 95° C. for 1 min; 30 cycles at 95° C. for 15 sec; and 68° C. for 5 min.

Analysis of the PCR products by electrophoresis on a 1.1% agarose gel, followed by staining with ethidium bromide, revealed that group A samples (enriched for low-abundance sequences) contained bands corresponding to IL6, P50, and IGFR sequences, whereas group B samples (negative control) contained no detectable bands corresponding to these sequences. The results indicate that the methods of the invention enrich for low-abundance transcripts of varying sizes.

### Example 4

### Preferred Subtractive Hybridization Method for Identifying Low-Abundance Polynucleotides that Differ Between a Test and Reference Sample

This example provides a protocol for a preferred embodiment of the general subtractive hybridization method of the invention. This embodiment employs a high-abundance enriched test polynucleotide pool and a low-abundance enriched reference polynucleotide pool, both produced by exemplary selection methods according to the invention.

A. Preparation of an Antisense Test RNA Pool that is Enriched in High-Abundance Sequences

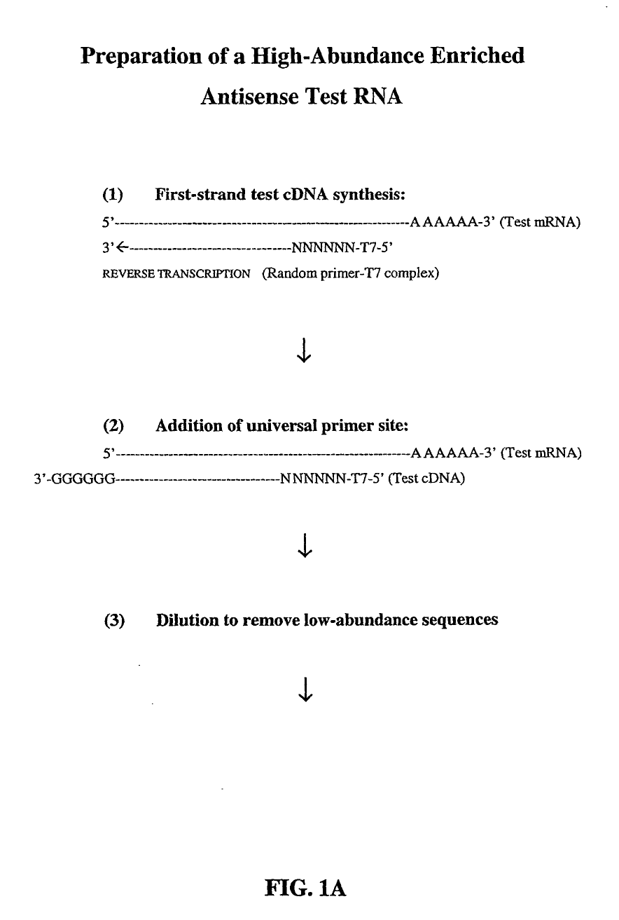

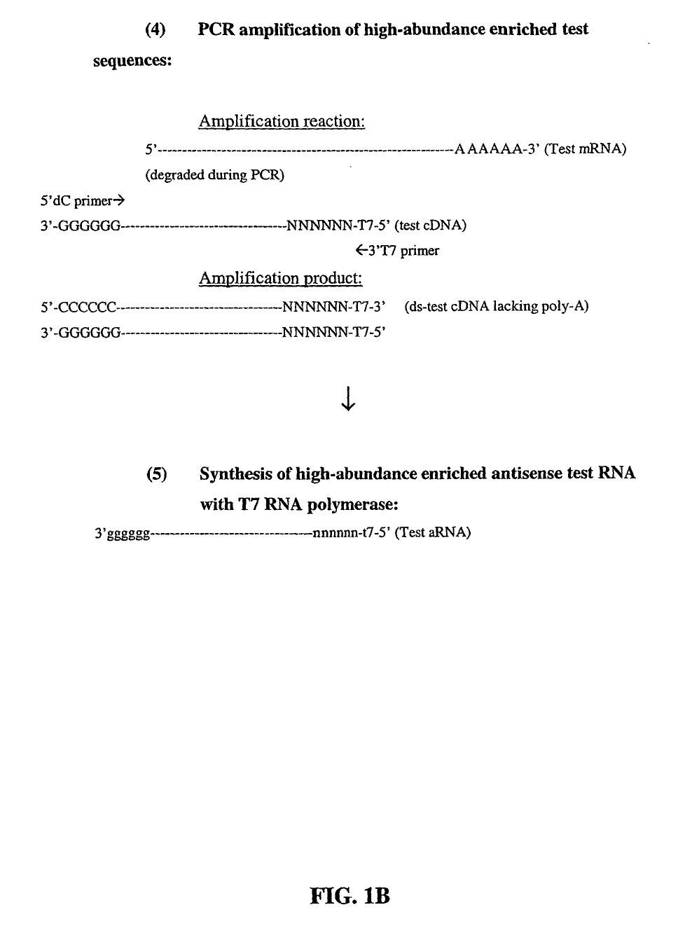

A high-abundance enriched antisense RNA pool is prepared from a test RNA sample as shown in FIG. 1 and described below.

1. First-strand (antisense) test cDNA is synthesized from a test RNA sample using reverse transcriptase and an antisense primer complex mixture. Each antisense primer complex includes a random sequence, which serves as the antisense primer. A T7 RNA promoter sequence is linked to the 5′ end of the random sequence.

2. A universal primer site is added to the 3′ end of the antisense test cDNA strand by oligonucleotide tailing. In this example, dG tailing is used to produce a poly-G tail.

3. The reaction mix of step 2 is serially diluted to remove low-abundance sequences. The removal of such sequences is monitored by PCR, as described in the above examples.

4. The diluted reaction mix is then subjected to PCR using a 5′ universal primer that hybridizes to the 3′ universal primer site of the newly synthesized antisense test cDNA strands. As shown in FIG. 1B (4), the 3′ primer is one that hybridizes to the T7 RNA promoter sequence at the 5′ end of the antisense test cDNA strands. PCR produces a pool of double-stranded test cDNA molecules that is enriched in high-abundance sequences.

5. Antisense test RNA is synthesized from the high-abundance enriched cDNA molecules using T7 RNA polymerase to produce driver for use in the first subtractive hybridization of procedure C (step C.1. below). This driver is enriched in high-abundance test RNA sequences.

B. Preparation of a Pool of Sense Reference cDNA Strands that is Enriched in Low-Abundance Sequences

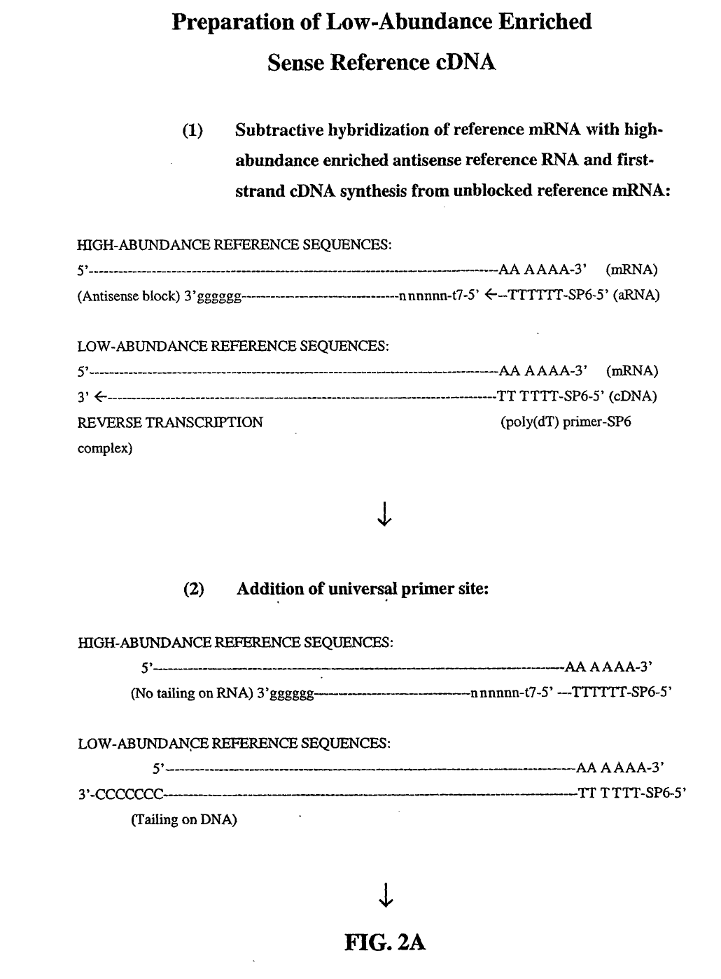

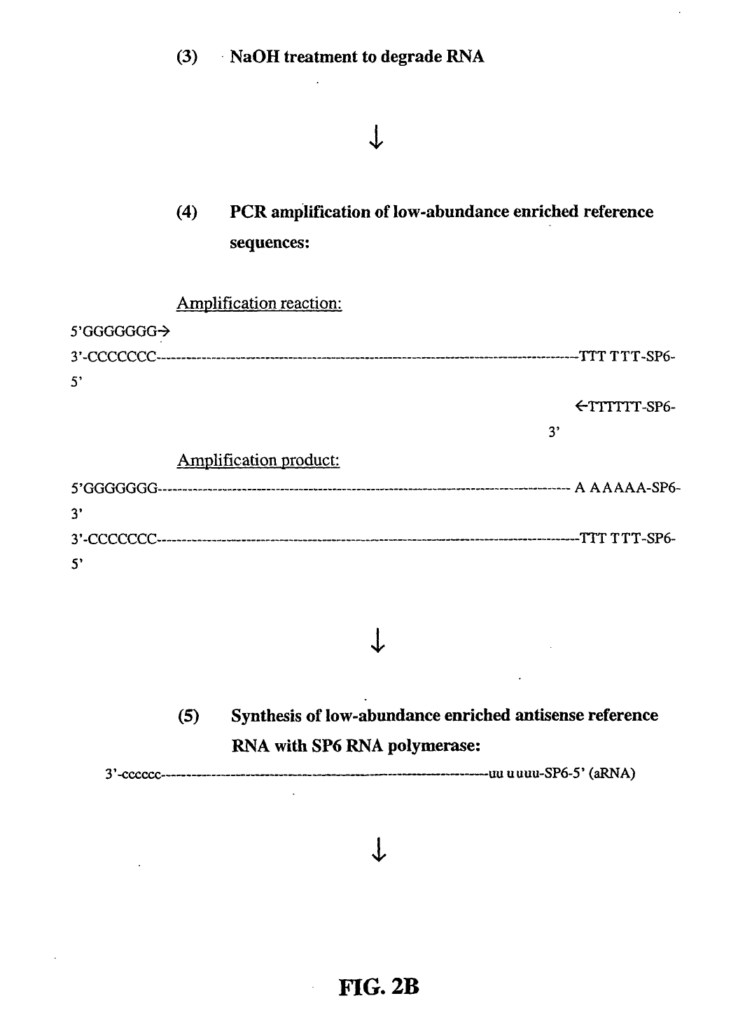

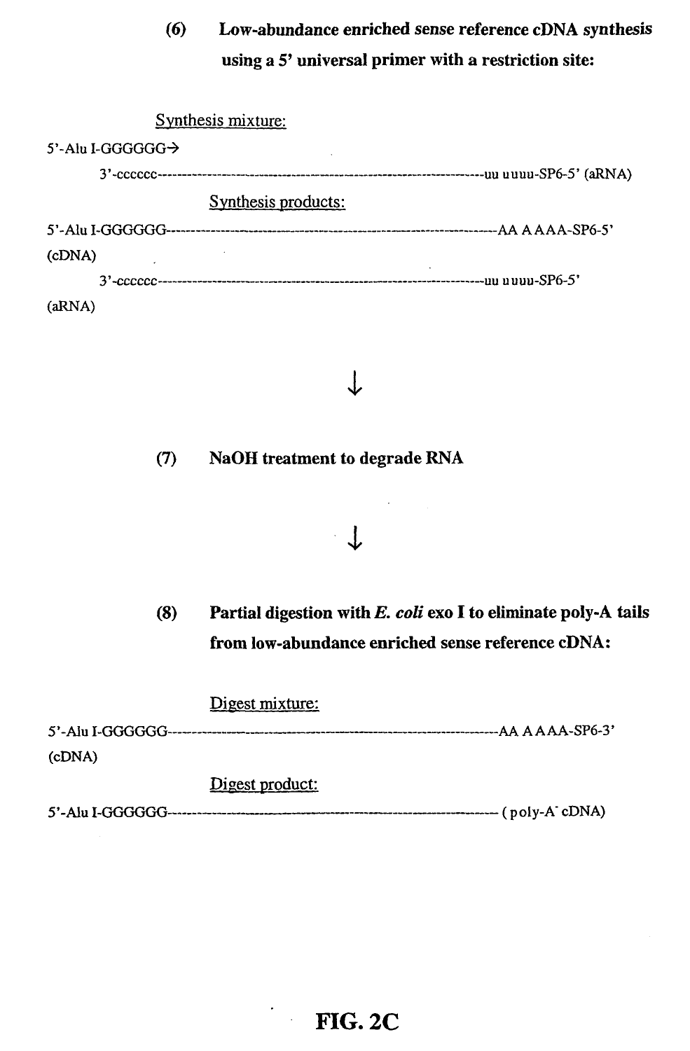

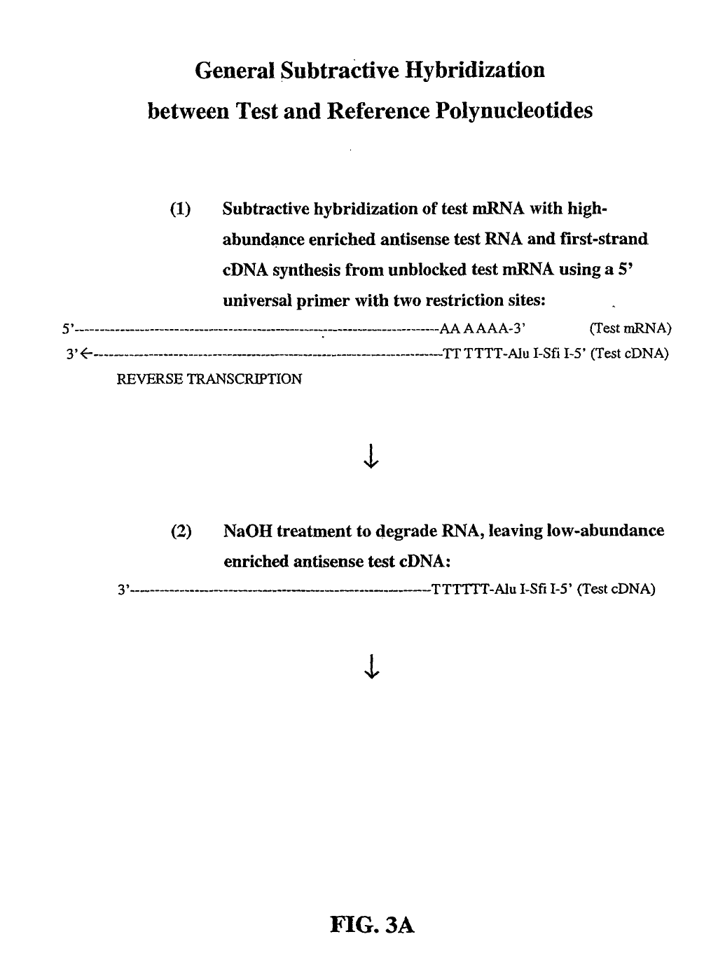

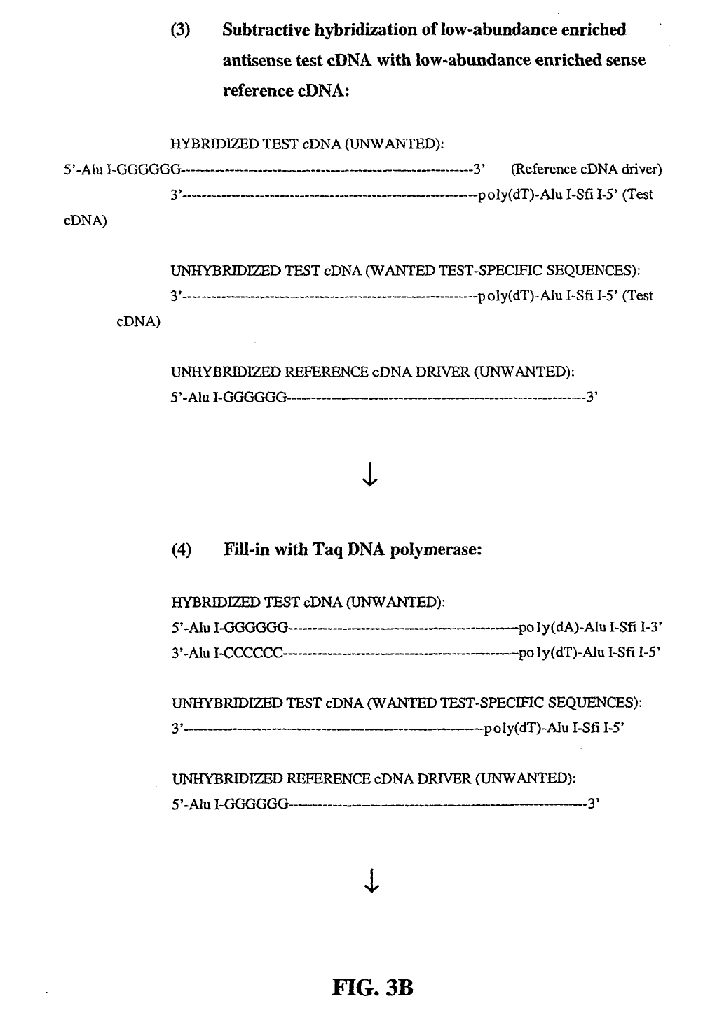

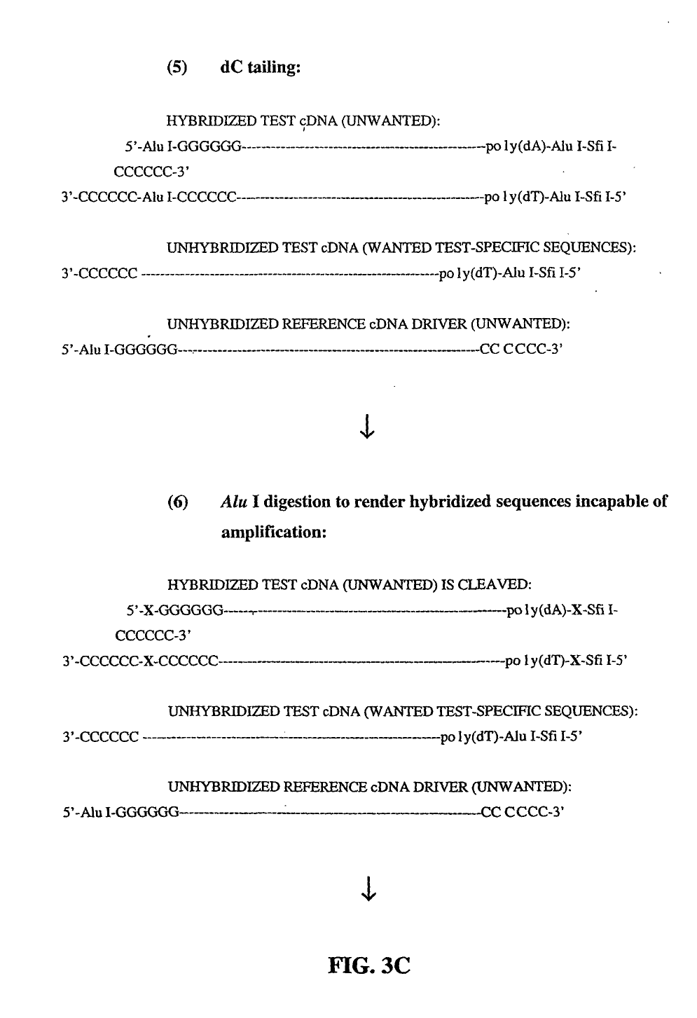

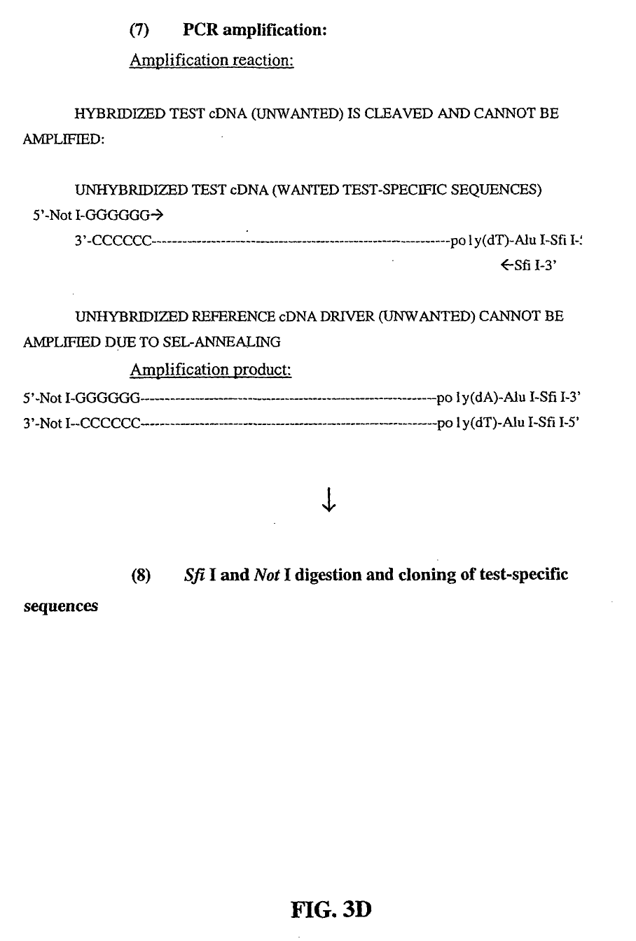

Antisense reference RNA is prepared as described for antisense test RNA in A.5. (above). This antisense reference RNA, which is enriched in high-abundance sequences, is used to produce a low-abundance enriched pool of sense reference cDNA strands as shown in FIG. 2 and described below.

1. The antisense reference RNA is used as driver in a subtractive hybridization reaction with reference mRNA to block the high-abundance mRNAs, leaving low-abundance reference mRNA molecules free to serve as a template for first-strand cDNA synthesis. First-strand (antisense) reference cDNA is synthesized using reverse transcriptase and an antisense primer complex including an oligo-dT sequence linked at the 5′ end to a SP6 RNA promoter sequence. (These primer and promoter sequences must be different from those used in A.1. above).

2. A universal primer site is added to the 3′ end of the antisense reference cDNA strands by oligonucleotide tailing. This universal primer site must be different from that added to the antisense test cDNA strands in A.2. above, and thus dC tailing is used to produce a polydc tail.

3. The reaction mixture of B.2. is then treated with 100 mM NaOH at 68° C. for 60 min. to degrade the reference mRNA, leaving the low-abundance enriched antisense reference cDNA strands.

4. The antisense reference cDNA strands are then subjected to PCR using a 5′ universal primer (here, poly-G) that hybridizes to the 3′ universal primer site and a 3′ antisense primer that hybridizes to that 5′ antisense primer site of the antisense reference cDNA strands (here, poly-A) linked to the SP6 promoter sequence). PCR produces a pool of double-stranded reference cDNA molecules that are enriched in low-abundance sequences.

5. Antisense reference RNA is synthesized from the low-abundance enriched reference cDNA molecules using SP6 polymerase.

6. Sense reference cDNA strands are synthesized from the antisense reference RNA using reverse transcriptase and a 5′universal primer that includes a restriction site at its 5′ end (e.g., Alu I linked to poly-G. This restriction site is one that is cleaved by an enzyme specific for double-stranded DNA. This feature is used to facilitate selective cleavage of hybridized sequences after the second hybridization reaction of the general hybridization method of C (below).

7. The reaction mixture of B.6. is then treated with 100 mM NaOH at 68° C. for 60 min. to degrade the antisense reference RNA, leaving sense reference cDNA strands.

8. The resulting sense reference cDNA strands are enriched in low-abundance sequences and can be used in the second subtractive hybridization reaction of the general subtractive hybridization method of C. To reduce non-specific hybridization between poly-A and poly-T sequences, the poly-A tails of the sense reference cDNA strands are removed by partial digestion with E. coli exonuclease I prior to their use in the second subtractive hybridization reaction of the invention. Suitable reaction conditions are determined empirically (e.g., by setting up multiple reactions for between about 5 and about 3 mins. and using monitoring poly-A removal by amplification using an oligonucleotide-dT pimer).

C. General Subtractive Hybridization between Test and Reference Polynucleotides

1. The antisense test RNA of A.5. is used as driver in a first subtractive hybridization reaction with test mRNA to block the high-abundance test mRNA molecules, leaving low-abundance test mRNA molecules free to serve as a template for first-strand cDNA synthesis. Low-abundance enriched first-strand (antisense) test cDNA is synthesized from the unhybridized test mRNA molecules using reverse transcriptase and an antisense primer including an oligo-dT sequence and two restriction sites 5′ of the oligo-dT sequence. (The primer sequence must be different from that used in B.2., but can be the same as or different from that used in A.1.) One restriction site (e.g., Alu I) is cleaved by an enzyme specific for double-stranded DNA. This feature is used to facilitate selective cleavage of hybridized sequences after the second hybridization reaction of the general hybridization method of C (below). The other restriction site is used to clone test-specific polynucleotides into a cloning vector after the second hybridization reaction.

2. The reaction mixture of C.2. is then treated with 100 mM NaOH at 68° C. for 60 min. to degrade the test mRNA, leaving low-abundance enriched antisense test cDNA strands.

3. The sense reference cDNA of B.7. is used as driver in a subtractive hybridization reaction with the antisense test cDNA strands of step 3 to block antisense test cDNA strands corresponding to polynucleotide sequences that are present in both the test and reference samples. This second hybridization reaction produces double-stranded cDNA molecules, unhybridized low-abundance enriched antisense test cDNA strands, and unhybridized low-abundance enriched sense reference cDNA strands (i.e., the driver). The hybrid duplexes correspond to sequences present in both the test and reference sample, whereas the unhybridized antisense test cDNA strands correspond to test-specific sequences.

4. The “sticky ends” of the hybrid duplexes are filled in using Taq polymerase to generate double-stranded Alu I restriction sites.

5. Oligonucleotide tailing is carried out with dC to add a poly-C tail to all molecules in the reaction mixture.

6. The hybrid duplexes are digested with Alu I, which cleave off the 3′ poly-C tails at either end of the duplex. This reaction leaves intact the unhybridized low-abundance enriched antisense test cDNA strands, and unhybridized low-abundance enriched sense reference cDNA strands (i.e., the driver). However, the driver has 5′ poly-G and 3′ poly-C sequences that self-anneal, preventing amplification.

7. Low-abundance enriched antisense test cDNA strands are converted to double-stranded test cDNA molecules by selective PCR using suitable primers. In this example, a 5′ primer includes poly-G, and a 3′ primer includes an Sfi I sequence that binds to the Sfi I sequence at the 5′ end of the antisense test cDNA strands. The 5′ primer also includes a restriction site at its 5′ end (here, Not I). PCR produces a pool of subtracted cDNA molecules that is enriched in low-abundance test sequences and in sequences that differ between the starting test and reference RNA samples. Each double-stranded molecule has a Not I restriction site at the 5′ end and a Sfi I restriction site at the 3′ end.

8. The mixture of step 7 is digested with Sfi I and Not I and ligated into a suitable vector to produce a library of low-abundance enriched, test-specific clones. SfiI and Not I are “infrequent cutters,” which ensures that almost all test-specific sequences remain intact, thus enhancing the recovery of full-length cDNAs. The use of two different restriction sites in this procedure facilitates directional cloning into expression vectors, followed by rapid analysis of the expressed genes.

What is claimed is:

1. A subtractive hybridization method for identifying one or more polynucleotides in a test sample that are absent from, or less abundant in, a reference sample, said method comprising:
a) providing high-abundance enriched polynucleotide strands of, or prepared from, a pool of test or reference polynucleotides that is enriched in high-abundance polynucleotide sequences relative to a test or reference polynucleotide sample, respectively;
b) contacting the high-abundance enriched polynucleotide strands with test polynucleotide strands of, or prepared from, the test polynucleotide sample under hybridization conditions to form a first hybridization mixture, thereby producing unhybridized test polynucleotide strands that are enriched in low-abundance polynucleotide sequences relative to the test polynucleotide sample;
c) synthesizing polynucleotide strands from the unhybridized test polynucleotide strands, thereby producing low-abundance enriched test polynucleotide strands;
d) providing low-abundance-enriched reference polynucleotide stands of, or prepared from, a reference pool of polynucleotides that is enriched in low-abundance polynucleotide sequences relative to the reference polynucleotide sample;
e) contacting the low-abundance enriched reference polynucleotide strands with the low-abundance enriched test polynucleotide strands under hybridization conditions to form a second hybridization mixture, thereby producing hybrid duplexes, unhybridized low-abundance enriched reference polynucleotide strands, and unhybridized low-abundance enriched test polynucleotide strands;
f) removing or digesting the hybrid duplexes; and
g) producing test-specific duplexes from the unhybridized low-abundance enriched test polynucleotide strands.

2. The method of claim 1 wherein the high-abundance enriched polynucleotide strands are of, or prepared from, a pool of test polynucleotides that is enriched in high-abundance polynucleotide sequences relative to a test polynucleotide sample.

3. The method of claim 1 wherein:
the high-abundance enriched polynucleotide strands of (a) comprise high-abundance enriched antisense polynucleotide strands;
the test polynucleotide strands of (b) comprise sense test polynucleotide strands;
the low-abundance enriched test polynucleotide strands of (c) comprise antisense test polynucleotide strands; and
the low-abundance enriched reference polynucleotide strands of (d) comprise sense reference polynucleotide strands.

4. The method of claim 3 wherein the antisense test polynucleotide strands are synthesized from the unhybridized sense test polynucleotide strands using a first antisense primer or a first antisense primer complex, said first antisense primer complex comprising a first antisense primer operably linked to a first RNA promoter sequence, wherein the first RNA promoter sequence is 5′ of the first antisense primer.

5. The method of claim 4 wherein the first antisense primer or first antisense primer complex comprises:
a) a sequence that binds to a primer site in the unhybridized sense test polynucleotide strands;
b) a first restriction site 5′ of the sequence of (a), wherein the first restriction site is cleaved by a restiction endonuclease that cleaves double-stranded polynucleotides, but leaves single-stranded polynucleotides substantially intact; and
c) a first universal primer site 5′ of the restriction site of (b).

6. The method of claim 5 wherein the antisense test polynucleotide strands are synthesized from the unhybridized sense test polynucleotide strands using a first antisense primer, and the first universal primer site of (c) comprises a second restriction site, wherein said second restriction site is different from said first restriction site.

7. The method of claim 5 wherein the sense reference polynucleotide strands comprise a third restriction site previously added to the 5′ ends of the sense reference polynucleotide strands, wherein the third restriction site is cleaved by a restriction endonuclease that cleaves double-stranded polynucleotides, but leaves single-stranded polynucleotides substantially intact.

8. The method of claim 1 wherein the hybrid duplexes are digested with at least one enzyme.

9. The method of claim 8 wherein the enzyme is a restriction endonuclease.

10. The method of claim 7 wherein the removal or digestion of the hybrid duplexes of (f) comprises:
i) treating the second hybridization mixture with an enzyme that renders single-stranded portions of the hybrid duplexes double-stranded, thereby producing double-stranded first, second, and third restriction sites in the hybrid duplexes;
ii) adding a second universal primer site to the 3′ ends of the polynucleotide strands in the second hybridization mixture; and
iii) treating the second hybridization mixture with one or more restriction endonuclease(s), wherein the restriction endonuclease(s) cleave(s) the hybrid duplexes at the double-stranded first and third restriction sites, but leaves the antisense test polynucleotide strands and the sense reference polynucleotide strands substantially intact.

11. The method of claim 10 wherein the test-specific duplexes are produced from the unhybridized antisense test polynucleotide strands by amplification.

12. The method of claim 11 wherein the test-specific duplexes are synthesized from the unhybridized antisense test polynucleotide stands using a first universal primer that hybridizes to the first universal primer site as the 3′ primer and a second universal primer that hybridizes to the second universal primer site as the 5′ primer.

13. The method of claim 11 wherein the amplification is performed by an enhanced polymerase chain reaction.

14. The method of claim 1 wherein the molar ratio of the high-abundance enriched polynucleotide strands to the test polynucleotide strands in the first hybridization mixture is between about 1 and about 100 to 1.

15. The method of claim 1 wherein the molar ratio of the low-abundance enriched reference polynucleotide strands to the low-abundance enriched test polynucleotide strands in the second hybridization mixture is between about 1 and about 100 to 1.

16. The method of claim 1 wherein the test and reference polynucleotide samples are mRNA samples.

17. The method of claim 3 wherein the high-abundance enriched antisense polynucleotide strands are antisense RNA molecules.

18. The method of claim 3 wherein the sense test polynucleotide strands are mRNA molecules.

19. The method of claim 3 wherein the antisense test polynucleotide strands are antisense cDNA strands.

20. The method of claim 3 wherein the sense reference polynucleotide strands are sense cDNA strands.

21. The method of claim 3 wherein the synthesis of the antisense test polynucleotide strands is primed using oligonucleotide-dT priming.

22. The method of claim 5 wherein the test and reference polynucleotide samples are test and reference mRNA samples and the high-abundance enriched antisense polynucleotide strands are test or reference antisense RNA molecules prepared by a method comprising:
a) synthesizing first antisense cDNA strands from the test or reference mRNA sample using a second antisense primer complex comprising a second antisense primer operably linked to a second RNA promoter sequence, wherein the second RNA promoter sequence is 5′ of the second antisense primer;
b) adding a third universal primer site to the 3′ ends of the first antisense cDNA strands;
c) diluting the first antisense cDNA strands to substantially eliminate at least some low-abundance antisense cDNA strands;
d) producing first double-stranded cDNA molecules from the remaining first antisense cDNA strands, wherein the first double-stranded cDNA molecules are enriched in high-abundance polynucleotide sequences relative to the starting mRNA sample; and
e) synthesizing high-abundance enriched antisense RNA molecules from the double-stranded cDNA molecules.

23. The method of claim 22 wherein the sense reference polynucleotides are sense cDNA strands prepared by a method comprising:
a) providing high-abundance enriched antisense RNA molecules of, or prepared from, a pool of reference polynucleotides that is enriched in high-abundance polynucleotide sequences relative to the reference polynucleotide sample;
b) contacting the high-abundance enriched antisense RNA molecules with the reference mRNA sample under hybridization conditions to form a hybridization mixture, thereby producing unhybridized mRNA molecules that are enriched in low-abundance polynucleotide sequences relative to the reference mRNA sample;
c) synthesizing second antisense cDNA strands from the unhybridized mRNA molecules using a third antisense primer or a third antisense primer complex, said third antisense primer complex comprising a third antisense primer operably linked to a third RNA promoter sequence, wherein the third RNA promoter sequence is 5′ of the third antisense primer;
d) adding a fourth universal primer site to the 3′ ends of the second antisense cDNA strands;
e) producing second double-stranded cDNA molecules from the second antisense cDNA strands;
f) synthesizing second antisense RNA molecules from the second double-stranded cDNA molecules; and
g) synthesizing first sense cDNA strands from the second antisense RNA molecules.

24. The method of claim 1 further comprising cloning at least one of the test-specific duplexes into a vector.

25. The method of claim 24 additionally comprising producing a polynucleotide library from said test-specific duplexes.

26. The method of claim 24 wherein the cloned test-specific duplex encodes a polypeptide, and the vector is an expression vector.

27. The method of claim 26 further comprising introducing the expression vector into a host cell and expressing the protein encoded by the cloned test-specific duplex.

28. The method of claim 4 wherein the test-specific duplexes are synthesized from the unhybridized antisense test polynucleotide strands using a first antisense primer complex, said method further comprising synthesizing one or more antisense RNA molecules from the test-specific duplexes.

29. The method of claim 28 additionally comprising introducing the one or more antisense RNA molecules synthesized from the test-specific duplexes into a cell.

30. The method of claim 29 wherein the cell is in vitro.

31. The method of claim 1 comprising employing one or more test-specific duplexes or one or more polynucleotides produced directly or indirectly therefrom in a hybridization reaction.

32. The method of claim 31 wherein at least one of the test-specific duplexes or a polynucleotide produced therefrom is labeled with a detectable label.

33. The method of claim 1 further comprising attaching a plurality of the test-specific duplexes or polynucleotides produced therefrom to a substrate to produce a polynucleotide array.

34. The method of claim 1 further comprising amplifying one or more of the test-specific duplexes.

35. The method of claim 34 wherein said one or more test-specific duplexes are amplified using one or more gene-specific primers.

36. The method of claim 1 wherein the test and reference polynucleotide samples are different samples.

37. The method of claim 36 wherein the test and reference polynucleotide samples are selected from one of the following:
mRNA from a first cell or tissue and mRNA from a second, different cell or tissue;
mRNA from a cell or tissue at a first stage of differentiation or development and mRNA from the cell or tissue at a second, different stage of differentiation or development;
mRNA from a cell or tissue treated with an active agent and mRNA from a cell or tissue that is untreated or treated with a second, different active agent; and
mRNA from a normal cell or tissue and mRNA from a diseased cell or tissue.

38. The method of claim 37 wherein the test and reference polynucleotide samples are mRNA from a normal cell or tissue and mRNA from a diseased cell or tissue, wherein the disease is an infectious disease or a cancer.

39. A method for functionally isolating single-stranded polynucleotides in a mixture of single- and double-stranded polynucleotides, said method comprising contacting a mixture of single- and double-stranded polynucleotides with one or more restriction endonucleases under conditions sufficient to allow digestion of double-stranded polynucleotides to a form that cannot serve as a template for a nucleotide synthesis reaction that uses the single-stranded polynucleotides as a template.

40. The method of claim 39 wherein said restriction endonuclease(s) cleave(s) a primer site from double-stranded polynucleotides in said mixture.

41. The method of claim 40 wherein uncleaved single-stranded polynucleotides are converted to double-stranded polynucleotides by amplification.

42. A plurality of polynucleotides prepared by subtractive hybridization between test and reference polynucleotide samples according to the method of claim 1, wherein the plurality of polynucleotides includes at least 10³ different polynucleotides and is substantially enriched in sequences that are:
either not present in the reference polynucleotide sample or are present in the reference polynucleotide sample in substantially lower concentration than in the test polynucleotide sample; and
low-abundance sequences, relative to the test polynucleotide sample.

43. The plurality of polynucleotides of claim 42 wherein the polynucleotides each comprise an RNA promoter sequence and a universal primer site.

44. The plurality of polynucleotides of claim 42 wherein the polynucleotides are double-stranded cDNA molecules.

45. The plurality of polynucleotides of claim 42 wherein the polynucleotides are antisense RNA molecules.

46. A kit comprising:
a) an antisense primer or antisense primer complex comprising:
i) a sequence that binds to a primer site in the unhybridized sense test polynucleotide strands;
ii) a first restriction site 5′ of the sequence of (i), wherein the first restriction site is cleaved by a restiction endonuclease that cleaves double-stranded polynucleotides, but leaves single-stranded polynucleotides substantially intact; and
iii) a first universal primer site 5′ of the restriction site of (iii); and
b) instructions for performing the method of claim 3.

47. A kit comprising:
a) the plurality of polynucleotides of claim 42;
b) an antisense primer complex comprising a antisense primer operably linked to an RNA promoter sequence, wherein the RNA promoter sequence is 5′ of the antisense primer; and
c) a sense primer.

48. A kit comprising:
a) the plurality of polynucleotides of claim 42; and
b) an RNA polymerase capable of transcribing antisense RNA from the plurality of polynucleotides.

49. A method for preparing a selected polynucleotide pool from a polynucleotide sample comprising:
a) synthesizing first antisense polynucleotide strands from sense polynucleotides of, or prepared from, the polynucleotide sample using an antisense primer complex comprising an antisense primer operably linked to an RNA promoter sequence, wherein the RNA promoter sequence is 5′ of the antisense primer;
b) adding a universal primer site to the 3′ ends of the first antisense polynucleotide strands;
c) diluting the first antisense polynucleotide strands to substantially eliminate at least some low-abundance first antisense polynucleotide strands; and
d) producing first double-stranded polynucleotides from the remaining first antisense polynucleotide strands, wherein the first double-stranded polynucleotides are enriched in high-abundance polynucleotide sequences relative to the polynucleotide sample.

50. The method of claim 49 wherein the polynucleotide sample is an mRNA sample, the first antisense polynucleotide strands are first antisense cDNA strands, and the first double-stranded polynucleotides are first double-stranded cDNA molecules.

51. The method of claim 50 wherein the synthesis of first antisense cDNA strands is primed using a random primer or an oligonucleotide-dT primer.

52. The method of claim 50 wherein the universal primer site is added to the 3′ end of the first antisense cDNA strands by template switching, oligonucleotide-tailing, or ligation.

53. The method of claim 49 wherein the RNA promoter sequence is an RNA promoter sequence recognized by a bacteriophage RNA polymerase selected from the group consisting of T7, T3, and SP6.

54. The method of claim 49 wherein the first double-stranded polynucleotides are produced by amplifying the remaining first antisense polynucleotide strands, and wherein the amplification is carried out using a universal primer that hybridizes to the universal primer site as the 5′ primer and using the antisense primer complex as the 3′ primer.

55. The method of claim 54 wherein the amplification is performed by enhanced polymerase chain reaction.

56. The method of claim 49 wherein the first double-stranded polynucleotides are produced using an enzyme mixture comprising a DNA polymerase, a DNA ligase, and an RNase.

57. The method of claim 49 further comprising synthesizing first antisense RNA molecules from the first double-stranded polynucleotides.

58. The method of claim 57 further comprising:
a) contacting the first antisense RNA molecules with sense polynucleotide strands of, or prepared from, the polynucleotide sample under hybridization conditions to form a hybridization mixture, thereby producing unhybridized sense polynucleotide strands that are enriched in low-abundance polynucleotide sequences relative to the polynucleotide sample;
b) synthesizing second antisense polynucleotide strands from the unhybridized sense polynucleotide strands using an antisense primer or an antisense primer complex, said antisense primer complex comprising an antisense primer operably linked to an RNA promoter sequence, wherein the RNA promoter sequence is 5′ of the antisense primer;
c) adding a universal primer site to the 3′ ends of the second antisense polynucleotide strands; and
d) producing second double-stranded polynucleotides from the second antisense polynucleotide strands.

59. The method of claim 58 wherein the molar ratio of the first antisense RNA molecules to the other polynucleotides in the hybridization mixture is between about 1 and about 100 to 1.

60. The method of claim 58 wherein the polynucleotide sample is an mRNA sample, the sense polynucleotide strands are the mRNA molecules in the mRNA sample, the second antisense polynucleotide strands are second antisense cDNA strands, and the second double-stranded polynucleotides are second double-stranded cDNA molecules.

61. The method of claim 60 wherein the synthesis of second antisense cDNA strands is primed using oligonucleotide-dT priming.

62. The method of claim 60 wherein the universal primer site is added to the 3′ end of the second antisense cDNA strands by template switching, oligonucleotide-tailing, or ligation.

63. The method of claim 58 wherein the second antisense polynucleotide strands are synthesized from the unhybridized sense polynucleotide strands using an antisense primer complex, and the RNA promoter sequence of (b) comprises an RNA promoter sequence for an RNA polymerase selected from the group consisting of T7, T3, and SP6.

64. The method of claim 58 wherein the second double-stranded polynucleotides are produced by amplifying the second antisense polynucleotide strands, and wherein the amplification is carried out using a universal primer that hybridizes to the universal primer site as the 5′ primer and using the antisense primer or antisense primer complex as the 3′ primer.

65. The method of claim 64 wherein the amplification is performed by enhanced polymerase chain reaction.

66. The method of claim 58 wherein the second double-stranded polynucleotides are produced using an enzyme mixture comprising a DNA polymerase, a DNA ligase, and an RNase.

67. The method of claim 58 wherein the universal primer and/or the antisense primer or antisense primer complex each comprise a restriction site.

68. The method of claim 58 wherein the second antisense polynucleotide strands are synthesized from the unhybridized sense polynucleotide strands using an antisense primer complex, said method further comprising synthesizing antisense RNA molecules from the second double-stranded polynucleotides.

69. The method of claim 58 comprising employing second double-stranded polynucleotides or a polynucleotide produced directly or indirectly therefrom in a hybridization reaction.

70. A method for preparing a selected polynucleotide pool from a polynucleotide sample comprising:
a) hybridizing first antisense polynucleotide strands prepared from a first polynucleotide sample to sense polynucleotide strands of, or prepared from, a second polynucleotide sample, wherein the first antisense polynucleotide strands are enriched in high-abundance polynucleotide sequences relative to the first polynucleotide sample, thereby producing unhybridized sense polynucleotide strands that are enriched in low-abundance polynucleotide sequences relative to the second polynucleotide sample; and
b) synthesizing second antisense polynucleotide strands from the unhybridized sense polynucleotide strands using an antisense primer or an antisense primer complex, said antisense primer complex comprising an antisense primer operably linked to an RNA promoter sequence, wherein the RNA promoter sequence is 5′ of the antisense primer;
c) adding a universal primer site to the 3′ ends of the second antisense polynucleotide strands;
d) producing double-stranded polynucleotides from the second antisense polynucleotide strands.

71. The method of claim 70 wherein the molar ratio of the first antisense polynucleotide strands to the other polynucleotides in the hybridization mixture is between about 1 and about 100 to 1.

72. The method of claim 70 wherein the first and second polynucleotide samples are mRNA samples, the sense polynucleotide strands are the mRNA molecules, the first antisense polynucleotide strands are antisense RNA, the second antisense polynucleotide strands are antisense cDNA strands, and the double-stranded polynucleotides are double-stranded cDNA molecules.

73. The method of claim 70 wherein the double-stranded polynucleotides are produced by amplifying the second antisense polynucleotide strands, and wherein the amplification is carried out using a universal primer that hybridizes to the universal primer site as the 5′ primer and using the antisense primer or antisense primer complex as the 3′ primer.

74. The method of claim 70 wherein the universal primer and/or the antisense primer or antisense primer complex each comprise a restriction site.

75. The method of claim 70 further comprising cloning at least one of the double-stranded polynucleotides into a vector.

76. The method of claim 75 wherein the cloned polynucleotide encodes a polypeptide, and the vector is an expression vector.

77. The method of claim 76 further comprising introducing the expression vector into a host cell and expressing the protein encoded by the cloned double-stranded polynucleotide.

78. The method of claim 70 wherein the second antisense polynucleotide strands are synthesized from the unhybridized sense polynucleotide strands using an antisense primer complex, said method further comprising synthesizing antisense RNA molecules from the double-stranded polynucleotides.

79. The method of claim 70 comprising employing one or more of the double-stranded polynucleotides or a polynucleotide produced directly or indirectly therefrom in a hybridization reaction.

80. The method of claim 79 wherein at least one of the double-stranded polynucleotides or a polynucleotide produced therefrom is labeled with a detectable label.

81. The method of claim 70 further comprising attaching a plurality of the double-stranded polynucleotides or polynucleotides produced therefrom to a substrate to produce a polynucleotide array.

82. The method of claim 70 further comprising amplifying at least one of the double-stranded polynucleotides.

83. The method of claim 82 wherein said one or more double-stranded polynucleotides are amplified using one or more gene-specific primers.

84. The method of claim 70 wherein the first and second polynucleotide samples are different samples.

85. A plurality of polynucleotides prepared from a polynucleotide sample, wherein the plurality of polynucleotides includes at least 103 different polynucleotides and is substantially enriched in high-abundance polynucleotide sequences relative to the polynucleotide sample, wherein the polynucleotides each comprise a RNA promoter sequence and a universal primer site.

86. A plurality of polynucleotides prepared from a polynucleotide sample, wherein the plurality of polynucleotides includes at least 10³ different polynucleotides and is substantially enriched in low-abundance polynucleotide sequences relative to the polynucleotide sample.

87. The plurality of polynucleotides of claim 86 wherein the polynucleotides each comprise a RNA promoter sequence and a universal primer site.

88. The plurality of polynucleotides of claim 86 wherein the polynucleotides are double-stranded cDNA.

89. The plurality of polynucleotides of claim 86 wherein the polynucleotides are antisense RNA.

90. A kit comprising:
a) an antisense primer complex comprising a antisense primer operably linked to an RNA promoter sequence, wherein the RNA promoter sequence is 5′ of the antisense primer;
b) a sense primer; and
c) instructions for performing the method of claim 49.

91. A kit comprising:
a) an antisense primer complex comprising a antisense primer operably linked to an RNA promoter sequence, wherein the RNA promoter sequence is 5′ of the antisense primer;
b) a sense primer; and
c) instructions for performing the method of claim 70.

92. A kit comprising:
a) the plurality of polynucleotide of claim 86;
b) an antisense primer complex comprising a antisense primer operably linked to an RNA promoter sequence, wherein the RNA promoter sequence is 5′ of the antisense primer; and
c) a sense primer.

93. A kit comprising:
a) the plurality of polynucleotides of claim 86; and
b) an RNA polymerase capable of transcribing antisense RNA from the plurality of polynucleotides.

94. A method for preparing a selected polynucleotide pool from a polynucleotide sample comprising:
a) synthesizing first antisense polynucleotide strands from sense polynucleotides of, or prepared from, the polynucleotide sample;
b) diluting the first antisense polynucleotide strands to substantially eliminate at least some low-abundance first antisense polynucleotide strands; and
c) producing first double-stranded polynucleotides from the remaining first antisense polynucleotide strands, wherein the first double-stranded polynucleotides are enriched in high-abundance polynucleotide sequences relative to the polynucleotide sample;
d) producing second antisense polynucleotide strands from the first double-stranded polynucleotides;
e) contacting the second antisense polynucleotide strands with sense polynucleotide strands of, or prepared from, the polynucleotide sample under hybridization conditions to form a hybridization mixture, thereby producing unhybridized sense polynucleotide strands that are enriched in low-abundance polynucleotide sequences relative to the polynucleotide sample;
f) synthesizing third antisense polynucleotide strands from the unhybridized sense polynucleotide strands;
g) producing second double-stranded polynucleotides from the third antisense polynucleotide strands.
